SEQUENCE LISTING (7 sequences)

SEQ ID NO: 1 - DNA, 1008 bp, Saccharomyces cerevisiae
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 2 - DNA, 1131 bp, Saccharomyces cerevisiae
atgtcgaagggaaaggttttgctggttctttacgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaacaagcaattaatggtgagtgggatattgccggcgtggctaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctggatgctcaaaagaggtacgctcagggagtaaagaacatcctaaatagttacttttccaaaaagtttgattaccgtccacaggatattattgtgcagaatggttcttatgccaccagagcttatggacagaagaaataa

SEQ ID NO: 3 - DNA, 1044 bp, Saccharomyces cerevisiae
atgactactgataccactgttttcgtttctggcgcaaccggtttcattgctctacacattatgaacgatctgttgaaagctggctatacagtcatcggctcaggtagatctcaagaaaaaaatgatggcttgctcaaaaaatttaataacaatcccaaactatcgatggaaattgtggaagatattgctgctccaaacgcctttgatgaagttttcaaaaaacatggtaaggaaattaagattgtgctacacactgcctccccattccattttgaaactaccaattttgaaaaggatttactaacccctgcagtgaacggtacaaaatctatcttggaagcgattaaaaaatatgctgcagacactgttgaaaaagttattgttacttcgtctactgctgctctggtgacacctacagacatgaacaaaggagatttggtgatcacggaggagagttggaataaggatacatgggacagttgtcaagccaacgccgttgccgcatattgtggctcgaaaaagtttgctgaaaaaactgcttgggaatttcttaaagaaaacaagtctagtgtcaaattcacactatccactatcaatccgggattcgtttttggtcctcaaatgtttgcagattcgctaaaacatggcataaatacctcctcagggatcgtatctgagttaattcattccaaggtaggtggagaattttataattactgtggcccatttattgacgtgcgtgacgtttctaaagcccacctagttgcaattgaaaaaccagaatgtaccggccaaagattagtattgagtgaaggtttattctgctgtcaagaaatcgttgacatcttgaacgaggaattccctcaattaaagggcaagatagctacaggtgaacctgcgaccggtccaagctttttagaaaaaaactcttgcaagtttgacaattctaagacaaaaaaactactgggattccagttttacaatttaaaggattgcatagttgacaccgcggcgcaaatgttagaagttcaaaatgaagcctaa

SEQ ID NO: 4 - DNA, 1083 bp, Saccharomyces cerevisiae
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 5 - DNA, 729 bp, Saccharomyces cerevisiae
atgctcctatttccaagaagaactaatatagcctttttcaaaacaacaggcatttttgctaattttcctttgctaggtagaaccattacaacttcaccatctttccttacacataaactgtcaaaggaagtaaccagggcatcaacttcgcctccaagaccaaagagaattgttgtcgcaattactggtgcgactggtgttgcactgggaatcagacttctacaagtgctaaaagagttgagcgtagaaacccatttggtgatttcaaaatggggtgcagcaacaatgaaatatgaaacagattgggaaccgcatgacgtggcggccttggcaaccaagacatactctgttcgtgatgtttctgcatgcatttcgtccggatctttccagcatgatggtatgattgttgtgccctgttccatgaaatcactagctgctattagaatcggttttacagaggatttaattacaagagctgccgatgtttcgattaaagagaatcgtaagttactactggttactcgggaaacccctttatcttccatccatcttgaaaacatgttgtctttatgcagggcaggtgttataatttttcctccggtacctgcgttttatacaagacccaagagccttcatgacctattagaacaaagtgttggcaggatcctagactgctttggcatccacgctgacacttttcctcgttgggaaggaataaaaagcaagtaa

SEQ ID NO: 6 - DNA, 1185 bp, Saccharomyces cerevisiae
atgtggacaaacactttcaaatggtgcagcaaaactgagaaggagaccactactgcagacgccaaggtgtgtgctagcgtgcagggcctgaaggccctgcagcagcaaattatggatagcaccactgtgcgtgggtccgttaataacacgatgactccaggcgggattaaccagtggcatttccacaataagcgtgcgaacaaagtgtgcacgcctacagtactaatccatggatacgctgcctcgtcgatggcgttctacaggacgtttgagaacctgtcagacaacattaaagatctatatgccatcgacttgccagccaatggtgcctcagaggcgccagcattgcaagtgaacaaaaccaagaagatcaagtctctcaggtttaagcatatagaagatgacgtagtaatccctgtgatagagaagcgccccccagcagaggacatcaagtcacacctggagcagtacgaaagctacttcgtagacaggatagagcaatggcggaaggataacaagcttcgcaaaataaacgtggtgggccattcattcggaggatatatttcattcaaatacgccctcaagtaccctgattccattgagaaactgtgtctcatatctcctcttggtgtggaaaacagtatacatgctatcacccacaaatgggagcccaacaccacctatccacttacgtttaccgacccatcgtcccgatattatacaagaaagctgaacgtaccgcggtttatctttgaaaaccagctaaacgttttgaagtggatgggacccataggctccaaattgtgctcgaactacatctccaccgcatacgtaaaagttccggatcagatatataaggactatcttttgcactcatttgtcgggaagaaccagacggtgcaaccacagacaatcaaggtgttcacccatttgtttgaaaggaatctgatcgctagagatccgataataaacaacgttcgttttttgaacccagcaacgcctgtcatgttcatgtatggcgagcacgattggatggataaatatgcgggctacttgactactgaatcaatgctaaaaaacaaggcaaaggctagctacgttgaagtccctgatgctggccataatcttttcctggacaacccgcagcactttgcctcttctttagtctcgttcctgtcaaagtaa

SEQ ID NO: 7 - DNA, 2010 bp, Saccharomyces cerevisiae
atgagtaatcctcaaaaagctctaaacgactttctgtccagtgaatctgttcatacacatgatagttctaggaaacaatctaataagcagtcatccgacgaaggacgctcttcatcacaaccttcacatcatcactctggtggtactaacaacaataataacaataataataataataataacagtaacaacaacaacaacggcaacgatgggggaaatgatgacgactatgattatgaaatgcaagattatagaccttctccgcaaagtgcgcggcctactcccacgtatgttccacaatattctgtagaaagtgggactgctttcccgattcaagaggttattcctagcgcatacattaacacacaagatataaaccataaagataacggtccgccgagtgcaagcagtaatagagcattcaggcctagagggcagaccacagtgtcggccaacgtgcttaacattgaagatttttacaaaaatgcagacgatgcgcataccatcccggagtcacatttatcgagaaggagaagtaggtcgagggctacgagtaatgctgggcacagtgccaatacaggcgccacgaatggcaggactactggtgcccaaactaatatggaaagcaatgaatcaccacgtaacgtccccattatggtgaagccaaagacattataccagaaccctcaaacacctacagtcttgccctccacataccatccaattaataaatggtcttccgtcaaaaacacttatttgaaggaatttttagccgagtttatgggaacaatggttatgattattttcggtagtgctgttgtttgtcaggtcaatgttgctgggaaaatacagcaggacaatttcaacgtggctttggataaccttaacgttaccgggtcttctgcagaaacgatagacgctatgaagagtttaacatccttggtttcatccgttgcgggcggtacctttgatgatgtggcattgggctgggctgctgccgtggtgatgggctatttctgcgctggtggtagtgccatctcaggtgctcatttgaatccgtctattacattagccaatttggtgtatagaggttttcccctgaagaaagttccttattactttgctggacaattgatcggtgccttcacaggcgctttgatcttgtttatttggtacaaaagggtgttacaagaggcatatagcgattggtggatgaatgaaagtgttgcgggaatgttttgcgtttttccaaagccttatctaagttcaggacggcaatttttttccgaatttttatgtggagctatgttacaagcaggaacatttgcgctgaccgatccttatacgtgtttgtcctctgatgttttcccattgatgatgtttattttgattttcattatcaatgcttccatggcttatcagacaggtacagcaatgaatttggctcgtgatctgggcccacgtcttgcactatatgcagttggatttgatcataaaatgctttgggtgcatcatcatcatttcttttgggttcccatggtaggcccatttattggtgcgttaatgggggggttggtttacgatgtctgtatttatcagggtcatgaatctccagtcaactggtctttaccagtttataaggaaatgattatgagagcctggtttagaaggcctggttggaagaagagaaatagagcaagaagaacatcggacctgagtgacttctcatacaataacgatgatgatgaggaatttggagaaagaatggctcttcaaaagacaaagaccaagtcatctatttcagacaacgaaaatgaagcaggagaaaagaaagtgcaatttaaatctgttcagcgcggcaaaagaacgtttggtggtataccaacaattcttgaagaagaagattccattgaaactgcttcgctaggtgcgacgacgactgattctattgggttatccgacacatcatcagaagattcgcattatggtaatgctaagaaggtaacatga

(19) United States
(12) Patent Application Publication
(10) Pub. No.: US 2023/0183721 A1
(43) Pub. Date: Jun. 15, 2023

(54) METHOD OF MODIFYING A YEAST STRAIN, MODIFIED YEAST STRAINS OBTAINED THEREBY AND USES THEREOF

(71) Applicant: STELLENBOSCH UNIVERSITY, Stellenbosch, WESTERN CAPE PROVINCE (ZA)

(72) Inventors: BIANCA ANINA BRANDT, STELLENBOSCH, WESTERN CAPE PROVINCE (ZA); JOHANN FERDINAND GÖRGENS, STELLENBOSCH, WESTERN CAPE PROVINCE (ZA); WILLEM HEBER VAN ZYL, STELLENBOSCH, WESTERN CAPE PROVINCE (ZA)

(21) Appl. No.: 17/923,659

(22) PCT Filed: May 5, 2021
(86) PCT No.: PCT/IB2021/053813

§ 371 Date: Nov. 7, 2022

(30) Foreign Application Priority Data
May 5, 2020 (ZA) 2020/02428

(51) Int. Cl. C12N 15/81; C12N 9/04; C12N 9/02; C12N 9/10

(52) U.S. Cl. CPC C12N 15/81; C12N 9/0006; C12N 9/0093; C12N 9/1029; C12N 9/1085; C12N 9/1022; C12Y 101/01001; C12Y 203/01051

(57) ABSTRACT

A method of producing a modified Saccharomyces cerevisiae yeast strain with enhanced resistance (or tolerance) to pretreatment-derived microbial inhibitors such as furans, phenolics and weak acids is provided, which comprises integrating at least one copy of the TAL1 gene and at least one copy of two or more of the FDH1, AR11 and ADH6 genes into the S. cerevisiae genome. A modified yeast strain so obtained is also provided, the modified yeast strain being capable of simultaneously overexpressing these genes relative to a yeast strain which hasn't been modified in the same manner. S. cerevisiae strains which have been modified as described herein can be used to ferment lignocellulosic hydrolysates containing pretreatment inhibitors such as furans, phenolics and weak acids. Suitable lignocellulosic hydrolysates include sugarcane bagasse (SCB) and waste streams from the pulp and paper industry, such as spent sulphite liquor (SSL).

### FIELD OF THE INVENTION

The invention relates to the production of Saccharomyces cerevisiae strains with enhanced tolerance towards microbial inhibitors. The modified S. cerevisiae strains are suitable for use in the production of biofuels, in particular for fermenting lignocellulosic hydrolysates such as sugarcane bagasse (SCB) and spent sulphite liquor (SSL) from the pulp and paper industry.

### BACKGROUND TO THE INVENTION

The increased pressure towards decreased carbon emissions has spurred the development of lignocellulose derived biofuels production as replacements for conventional fossil fuels. Fermentation serves as bioconversion to alcohols of sugar hydrolysates derived from polysaccharide-rich lignocellulose biomass. However, a major challenge linked to hydrolysis-fermentation of lignocellulose biomass is the recalcitrant nature of the material to biological (enzymatic) conversion (hydrolysis). Physicochemical pre-treatment is thus required to disrupt the compact crystalline structure and allow enzymatic access to the polysaccharides within, to generate fermentable sugars. The majority of such pre-treatment methods result in significant quantities of degradation products being formed, which have inhibitory effects on subsequent biological conversions.

Saccharomyces cerevisiae cannot naturally utilize xylose, the most abundant pentose sugar within lignocellulosic hydrolysates. Although co-fermentation of glucose and xylose remains a challenge, advances in strain development have resulted in the development of industrial S. cerevisiae strains with xylose utilizing capacity. Through metabolic engineering, heterologous xylose catabolic pathways such as the fungal oxidoreductive pathway (XR-XDH) and a bacterial xylose isomerase (XI) have been introduced into S. cerevisiae strains, individually and in combination. Recently, the industrial strain Cellux™1, a xylose engineered strain with a XI pathway, has been developed for the co-fermentation of glucose and xylose.

Fermentation strains are subjected to various microbial stresses during lignocellulose bioconversion, including microbial inhibitory compounds generated during physicochemical pre-treatment of lignocelluloses. Furans (degradation products of sugars), phenolics (derived from solubilizing lignin) and weak acids (such as acetic and formic acids) are formed during furan degradation and/or de-acetylation of hemicellulose. Such microbial inhibitors negatively impact the growth, fermentation and the xylose utilisation ability of yeast, which results in sub-optimal ethanol productivity and yields. Thus, microbial inhibitor toxicity represents a bottle-neck in lignocellulosic bioethanol production and negating these detrimental inhibitory effects is a fundamental challenge.

There is therefore a need for xylose-capable S. cerevisiae strains that are able to perform in the presence of microbial inhibitory stresses, particularly during fermentation of lignocellulose hydrolysates.

### SUMMARY OF THE INVENTION

According to a first embodiment of the invention, there is provided a method of producing a modified Saccharomyces cerevisiae yeast strain with enhanced resistance to furans, phenolics and weak acids, the method comprising the step of integrating at least one copy of the TAL1 gene and at least one copy of two or more of the FDH1, ARI1 and ADH6 genes into the S. cerevisiae genome, so that the modified yeast strain overexpresses these genes relative to a yeast strain which hasn't been modified in the same manner.

The method may comprise further integrating at least one copy of either or both of the PAD1 and ICT1 genes into the S. cerevisiae genome.

The method may further include the step of at least partially or completely deleting the FPS1 gene in the S. cerevisiae genome.

Multiple copies of some or each of the genes may be introduced into the S. cerevisiae genome. Each of the integrated genes may be under the control of a constitutive promoter.

The strain may be an S. cerevisiae strain with xylose utilizing capacity.

The method may comprise the step of integrating at least one copy of the TAL1 and FDH1 genes and either or both of the ARI1 and ADH6 genes into the S. cerevisiae genome.

The method may comprise integrating at least one copy of each of TAL1+FDH1+ARI1 into the S. cerevisiae genome.

The method may comprise integrating at least one copy of each of TAL1+FDH1+ADH6 into the S. cerevisiae genome.

The method may comprise integrating at least one copy of each of TAL1+FDH1+ARI1+ADH6 into the S. cerevisiae genome.

The method may comprise integrating at least one copy of each of TAL1+FDH1+ARI1+either or both of PAD1 and ICT1 into the S. cerevisiae genome.

The method may comprise integrating at least one copy of each of TAL1+FDH1+ADH6+either or both of PAD1 and ICT1 into the S. cerevisiae genome.

The method may comprise integrating at least one copy of each of TAL1+FDH1+ARI1+ADH6+either or both of PAD1 and ICT1 into the S. cerevisiae genome.

The method may comprise integrating at least one copy of each of TAL1+ADH6+ARI1 into the S. cerevisiae genome.

The method may comprise integrating at least one copy of each of TAL1+ADH6+FDH1 into the S. cerevisiae genome.

The method may comprise integrating at least one copy of each of TAL1+ADH6+ARI1+FDH1 into the S. cerevisiae genome.

The method may comprise integrating at least one copy of each of TAL1+ADH6+ARI1+FDH1+either of PAD1 and ICT1 or both of PAD1 and ICT1 into the S. cerevisiae genome.

The method may comprise integrating at least one copy of each of TAL1+ADH6+FDH1 either of PAD1 and ICT1 or both of PAD1 and ICT1 into the S. cerevisiae genome.

The method may comprise integrating at least one copy of each of TAL1+ADH6+ARI1+either of PAD1 and ICT1 or both of PAD1 and ICT1 into the S. cerevisiae genome.

The genes may be integrated sequentially.

The genes may be integrated in any order or in the order as listed above.

According to a further embodiment of the invention, there is provided a double gene expression cassette comprising the TAL1 and FDH1 genes.

According to a further embodiment of the invention, there is provided a double gene expression cassette comprising the ARI1 and ADH6 genes.

According to a further embodiment of the invention, there is provided a double gene expression cassette comprising the PAD1 and ICT1 genes.

According to a further embodiment of the invention, there is provided a double gene expression cassette comprising the TAL1 and ADH6 genes.

According to a further embodiment of the invention, there is provided a double gene expression cassette comprising the ARI1 and FDH1 genes.

The TAL1, ARI1 and PAD1 genes may be under the control of the PGK1 gene promoter.

The FDH1, ADH6 and ICT1 genes may be under the control of the ENO1 gene promoter.

According to a further embodiment of the invention, there is provided a Saccharomyces cerevisiae yeast strain which has been modified to overexpress at least the TAL1 gene and at least one copy of two or more of the FDH1, ARI1 and ADH6 genes relative to an unmodified strain, wherein the modified strain has increased resistance to furans, phenolics and weak acids compared to the unmodified strain.

The modified strain may further overexpress one or both of the PAD1 and ICT1 genes relative to an unmodified strain.

The FPS1 gene may be partially or completely deleted in the modified strain.

The modified strain may comprise multiple integrated copies of the genes which are overexpressed.

Each of the integrated genes may be under the control of a constitutive promoter.

The strain may be an S. cerevisiae strain with xylose utilizing capacity.

The modified strain may comprise integrated copies of any of the following genes:
TAL1+FDH1+ARI1;
TAL1+FDH1+ADH6;
TAL1+FDH1+ARI1+ADH6;
TAL1+FDH1+ARI1+either or both of PAD1 and ICT1;
TAL1+FDH1+ADH6+either or both of PAD1 and ICT1;
TAL1+FDH1+ARI1+ADH6+either or both of PAD1 and ICT1;
TAL1 ADH6 ARI1;
TAL1 ADH6 FDH1;
TAL1 ADH6 ARI1 FDH1;
TAL1 ADH6 ARI1 FDH1+either of PAD1 and ICT1 or both of PAD1 and ICT1;
TAL1 ADH6 FDH1 either of PAD1 and ICT1 or both of PAD1 and ICT1; or
TAL1 ADH6 ARI1+either of PAD1 and ICT1 or both of PAD1 and ICT1.

According to a further embodiment of the invention, there is provided a method of fermenting a lignocellulosic hydrolysate, the method comprising using a modified yeast as described above to ferment sugars in the lignocellulosic hydrolysate.

The lignocellulosic hydrolysate may be sugarcane bagasse (SCB) or may be a waste stream from the pulp and paper industry, such as spent sulphite liquor (SSL).

### BRIEF DESCRIPTION OF THE FIGURES

FIG. 1: shows a graphical illustration of the sequential integration of gene combinations into existing yeast strains to obtain derived yeast strains designated as TFA7, AP1 and TP1.

FIG. 2: shows the performance of final transformants in (A) 2% SC-X supplemented with 65% v/v sugarcane hydrolysate spiked with 20 g L⁻¹ furfural and formic acid, with a straight line indicating benchmark performance of Cellux™1 parent and (B) the % conversion of inhibitors. (C) A spot chart illustrates transformant performance to various inhibitors/stressors at OD₆₀₀ (from left to right) 5, 1, 0.5, 0.1 and 0.01 in 2% SC only or supplemented with 1 g L⁻¹ furfural, 6 g L⁻¹ acetic acid and 0.8 g L⁻¹ formic acid for weak acids, or 0.6 g L⁻¹ syringaldehyde, and growth at 37° C.

FIG. 3: shows the performances of final transformants in 2% SC-X fermentations supplemented with either, (A) 1 g L⁻¹ cinnamic acid, (B) 5 g L⁻¹ furfural or (C) 6 g L⁻¹ acetic and 1 g L⁻¹ formic acid. (D) The ethanol yields of various strains in 2% SC-X fermentations supplemented with 5 g L⁻¹ furfural at 48 versus 72 h.

FIG. 4: shows the % conversion of 5 g L⁻¹ furfural and 0.8 g L⁻¹ formic acid by transformants in 2% SC fermentations supplemented with synthetic IC with (A) glucose and xylose as carbon source or (B) xylose as only carbon source at 120 h.

FIG. 5: compares the fermentation capacity of steam exploded and undetoxified sugarcane bagasse whole slurry hydrolysate using S. cerevisiae TP-1 and S. cerevisiae CelluX™4. (A,B)—Glucose, xylose, ethanol and acetate time-profiles during the fermentation of the whole slurry hydrolysate, (C,D)—Furfural and 5-hydroxymethylfurfural profiles during the fermentation of the whole slurry hydrolysate, (E) comparison of the phenolic acid and phenolic aldehyde concentrations after fermentation with the transformant strain TP-1 and CelluX™4 relative to the initial whole slurry hydrolysate. Fermentations were performed with an initial inoculum of 1.5 g CDW.L⁻¹ at pH 5.5, 30 C and a shaking speed of 150 rpm for 120 h. All hydrolysates were supplemented with 0.5% (w/w) corn steep liquor.

FIG. 6: shows the DNA sequence of the TAL1 gene (SEQ ID NO: 1).

FIG. 7: shows the DNA sequence of the FDH1 gene (SEQ ID NO: 2).

FIG. 8: shows the DNA sequence of the ARI1 gene (SEQ ID NO: 3).

FIG. 9: shows the DNA sequence of the ADH6 gene (SEQ ID NO: 4).

FIG. 10: shows the DNA sequence of the PAD1 gene (SEQ ID NO: 5).

FIG. 11: shows the DNA sequence of the ICT1 gene (SEQ ID NO: 6).

FIG. 12: shows the DNA sequence of the FPS1 gene (SEQ ID NO: 7).

FIG. 13: shows the weight loss (g) of (A) TFA and (B) TAF transformants in 50% v/v SSL over 120 h. The horizontal line indicated the weight loss threshold of CelluX™4 at 120 h. Data points indicate average of triplicate samples.

FIG. 14: shows fermentation of sugarcane hydrolysate by (A) C4TP1 transformants and (B) C4TP3 transformants, over 88 h. All fermentations were conducted in triplicate and data points indicate the average of triplicate. Errors bars indicate standard deviation within triplicate.

FIG. 15: shows xylose utilization of transformants over 24 h in 2% YPX medium. Fermentation progression was recorded as weigh loss (g). All fermentations were conducted in triplicate and data points indicate the average of triplicate. Errors bars indicate standard deviation within triplicate.

### DETAILED DESCRIPTION OF THE INVENTION

A method of producing a modified Saccharomyces cerevisiae yeast strain with enhanced resistance (or tolerance) to pretreatment-derived microbial inhibitors such as furans, phenolics and weak acids is provided, the method comprising integrating at least one copy of the TAL1 gene and at least one copy of two or more of the FDH1, ARI1 and ADH6 genes into the S. cerevisiae genome. A modified yeast strain so obtained is also provided, the modified yeast strain being capable of simultaneously overexpressing these genes relative to a yeast strain which hasn't been modified in the same manner.

Throughout the specification and claims unless the context requires otherwise the word “comprise” or variations such as “comprises” or “comprising” will be understood to imply the inclusion of a stated integer or group of integers but not the exclusion of any other integer or group of integers.

The expression “yeast strain” denotes a relatively homogeneous population of yeast cells.

Any Saccharomyces cerevisiae strain may be modified by the method described herein.

The method optionally includes further integrating at least one copy of one or both of the PAD1 and ICT1 genes into the S. cerevisiae genome.

These 6 genes are endogenous to S. cerevisiae. Each of the integrated genes is under the control of a constitutive promoter, such as the PGK1 gene promoter for TAL1, ARI1 and PAD1 and the ENO1 promoter for FDH1, ADH6 and ICT1.

The modified strain can comprise integrated copies of at least the following genes:
TAL1+FDH1+ARI1;
TAL1+FDH1+ADH6;
TAL1+FDH1+ARI1+ADH6;
TAL1+FDH1+ARI1+either of PAD1 and ICT1 or both of PAD1 and ICT1;
TAL1+FDH1+ADH6+either of PAD1 and ICT1 or both of PAD1 and ICT1;
TAL1+FDH1+ARI1+ADH6+either of PAD1 and ICT1 or both of PAD1 and ICT1;
TAL1+ADH6+ARI1;
TAL1+ADH6+FDH1;
TAL1+ADH6+ARI1+FDH1; or
TAL1+ADH6+ARI1+FDH1+either of PAD1 and ICT1 or both of PAD1 and ICT1.

The TAL1 gene (NM_001182243) encodes a transaldolase. The gene may have a DNA sequence that is at least 90%, at least 95%, at least 96%, at least 97%, at least 98%, at least 99% or identical to SEQ ID NO: 1.

The FDH1 gene (NM_001183808) encodes a formate dehydrogenase. The gene may have a DNA sequence that is at least 90%, at least 95%, at least 96%, at least 97%, at least 98%, at least 99% or identical to SEQ ID NO: 2.

The ARI1 gene (NM_001181022) encodes a NADPH-dependent aldehyde reductase. The gene may have a DNA sequence that is at least 95%, at least 96%, at least 97%, at least 98%, at least 99% or identical to SEQ ID NO: 3.

The ADH6 gene (NC_001145) encodes an alcohol dehydrogenase. The gene may have a DNA sequence that is at least 90%, at least 95%, at least 96%, at least 97%, at least 98%, at least 99% or identical to SEQ ID NO: 4.

The PAD1 gene (NM_001180846) encodes a phenylacrylic acid decarboxylase. The gene may have a DNA sequence that is at least 90%, at least 95%, at least 96%, at least 97%, at least 98%, at least 99% or identical to SEQ ID NO: 5.

The ICT1 gene (NC_001144) encodes lysophosphatidic acid acyltransferase. The gene may have a DNA sequence that is at least 90%, at least 95%, at least 96%, at least 97%, at least 98%, at least 99% or identical to SEQ ID NO: 6.

The FPS1 aquaglyceroporin gene (NM_001181863) encodes a channel protein responsible for glycerol efflux and intake of acetic acid.

The genes can be integrated by homologous recombination of delta integration cassettes into native delta sequences in the parent S. cerevisiae genome, although any other method of modifying the parent strain to overexpress the genes can also be used. Multiple copies of some or each of these genes can be introduced into the S. cerevisiae genome using these methods.

The genes can be integrated in any order. Alternatively, the genes can be integrated in the order listed above.

The genes can be integrated in pairs. Alternatively, the genes can be integrated in any other suitable manner.

In one embodiment, a first double gene expression cassette including the TAL1 and FDH1 genes and a second double gene expression cassette including the ARI1 and ADH6 genes are sequentially integrated into the parent genome.

In another embodiment, a first double gene expression cassette including the TAL1 and ADH6 genes and a second double gene expression cassette including the ARI1 and ADH6 genes are sequentially integrated into the parent genome.

In another embodiment, a first double gene expression cassette including the TAL1 and FDH1 genes, a second double gene expression cassette including the ARI1 and ADH6 genes and a third double gene expression cassette including the PAD1 and ICT1 genes are sequentially integrated into the parent genome.

In another embodiment, a first double gene expression cassette including the TAL1 and ADH6 genes, a second double gene expression cassette including the ARI1 and ADH6 genes and a third double gene expression cassette including the PAD1 and ICT1 genes are sequentially integrated into the parent genome.

Single delta plasmids can optionally be used to perform the above steps.

The FPS1 gene in the S. cerevisiae genome can be at least partially silenced (e.g. by at least one gene knock of FPS1), or can be completely silenced, in any of the modified strains.

The strain is typically a S. cerevisiae strain with xylose utilizing capacity.

S. cerevisiae strains which have been modified as described herein can be used to ferment lignocellulosic hydrolysates containing pretreatment inhibitors such as furans (e.g. furan aldehydes, furfural, 5-hydroxymethylfurfural), phenolics (e.g. vanillin, syringaldehyde, coniferal aldehyde, 3,4-dihydrobenzoic acid, vanillic acid, syringic acid, ferulic acid, p-coumaric acid) and weak acids (e.g. acetic acid and formic acid). Suitable lignocellulosic hydrolysates include sugarcane bagasse (SCB) and waste streams from the pulp and paper industry, such as spent sulphite liquor (SSL). The fermentation process may be used to ferment sugars to ethanol or to remove sugars from the lignocellulosic hydrolysate. Preferably, the modified S. cerevisiae strains achieve higher metabolic yields, ethanol yields and/or volumetric productivities than the parent strain.

In another embodiment of the invention, one or more modified strains comprising integrated copies of at least the following genes are provided:
ARI1+ADH6+TAL1;
ARI1+ADH6+FDH1;
ARI1+ADH6+TAL1+FDH1;
ARI1+ADH6+TAL1+either of PAD1 and ICT1 or both of PAD1 and ICT1;
ARI1+ADH6+FDH1+either of PAD1 and ICT1 or both of PAD1 and ICT1; or
ARI1+ADH6+TAL1+FDH1+either of PAD1 and ICT1 or both of PAD1 and ICT1.

The FPS1 gene in the S. cerevisiae genome of any of these modified strains can be at least partially silenced (e.g. by at least one gene knock of FPS1), or can be completely silenced.

In studies performed by the applicant, industrial S. cerevisiae strains with enhanced tolerance towards pretreatment-derived microbial inhibitors were developed by identifying gene combinations that confer yeast resistance to multiple inhibitors (thus cumulative inhibitor resistance phenotype) with minimum impact on the xylose fermentation ability. The strategy consisted of sequential delta-integration of double gene cassettes containing one gene conferring broad inhibitor tolerance (ARI1, PAD1 or TAL1) coupled with an inhibitor specific gene (ADH6, FDH1 or ICT1). One copy of the FPS1 gene in a xylose capable (XI) industrial strain S. cerevisiae (CelluX™1 and CelluX™4) was deleted, and the TAL1, PAD1, FDH1, ICT1, ARI1 and ADH6 genes were overexpressed to develop a range of multi-inhibitor resistant strains. The impact of gene combinations on the development of cumulative inhibitor resistance phenotypes was evaluated.

Partial deletion of the FPS1 in the CelluX™1 strain resulted in a moderate increase in the ethanol yield (˜5% increment), which could be ascribed to the partial deletion and/or differences in the genetic background of the strain and media (carbon source: glucose and xylose; concentration of acetic acid 2.9 g/L, pH 5).

CelluX™1ΔFPS1-C5 transformants were used as background for the sequential delta integration of nine different gene combinations (see examples). These combinations were assessed in terms of ethanol yield and biomass formation (growth measured at OD₆₀₀) during SSCF fermentations supplemented with 65% v/v sugarcane pretreatment liquor. Compared to parental strains, there was generally a trade-off between the growth and the ethanol yield of the transformants from the first round of integration. Nevertheless, some combinations of genes resulted in significant improvement in ethanol yield (ARI1-ADH6 and TAL1-FDH1) while other combinations were detrimental for both parameters (TAL1-ICT1).

None of the transformants screened could ferment the sugars when the pretreatment liquor concentration in the media was increased from 65 to 75% v/v (data not shown), which suggests that selecting 2 genes to improve inhibitor resistance fell short of multi-inhibitor resistance phenotypes. However, the inhibitor specific combinations AA (ARI1+ADH6) for furans and TF (TAL1+FDH1) for weak acids improved both ethanol yields and biomass in 65% v/v pretreatment liquor fermentations. There could be a function for inhibitor specific combinations whereby different inhibitor specific combinations in tandem could have a cumulative effect in constructing multi-resistance phenotypes. The AA and TF combinations were thus the baseline for further strain development, which resulted in strains with these specific gene combinations, but in alternate integration sequences. The PI (PAD1+ ICT1) combination was included only for the final integration as it did not improve ethanol yields but did improve growth (Table 2).

The second round of integrations also resulted in a trade-off between ethanol yield and biomass growth, with the ATF transformants more prone towards biomass (2-17% increment in 70.8% of the transformants) while the TFA transformants were more inclined to ethanol yield improvement (5-24% increment in 58.3% of the transformants). It was also found that transformants presented phenotypic plasticity, with no differences in growth or ethanol yield compared to parental strain when the toxicity of the media was reduced. The selected transformant for the next round of integration, TFA7, was also able to grow in syringaldehyde at low cells concentration (FIG. 2C).

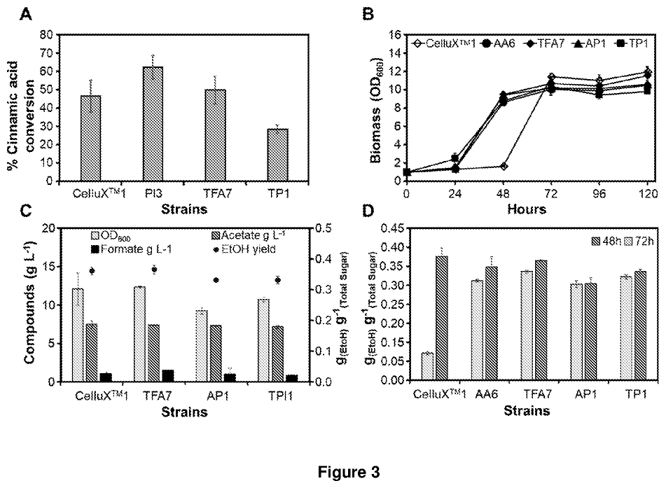

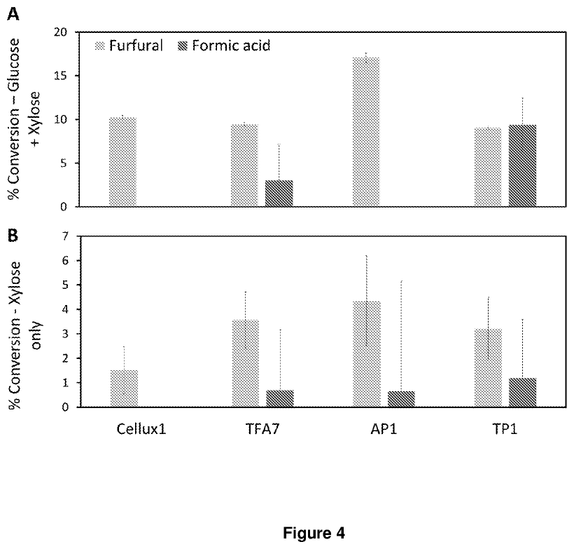

The third round of integration resulted in transformants with the combination of the selected six genes in a partial FPS1 gene deletion background. Of particular interest here was whether subsequent additions of inhibitor-specific gene combinations had a cumulative effect or build-up towards multi-inhibitor resistance phenotypes. The performance of the final transformants was evaluated during fermentations supplemented with inhibitors (single inhibitor or in a cocktail) and different sources of sugars. The transformants were subjected to fermentations with synthetic inhibitor cocktails (IC), as this allowed for a more controlled assessment whereby phenotypes for specific inhibitor resistance could be linked to specific gene combinations. Compared to the parental strain, these transformants showed improved growth during fermentations supplemented with 65% v/v pretreatment liquor (FIG. 2A) and proved to be able to detoxify furfural and formic acid despite their high concentration in the media (20 g/L) (FIG. 2B). Final transformants presented strong furan resistance phenotypes with a 24 h reduction of the lag phase in synthetic media containing only furfural (FIG. 3B), and improved conversion of furfural when the fermentations were carried out in a mixed synthetic inhibitor cocktail (IC) with glucose and xylose (AP1, FIG. 4A).

The inclusion of the PAD1-ICT1 combination in both the first and the third round of integration seemed to increase the sensitivity of the yeast towards weak acids at concentrations found in the sugarcane bagasse pretreatment liquor, as inferred from the inhibitor tolerance assays (FIG. 2C) and drop in growth in synthetic inhibitor media containing only weak acids (FIG. 2C). This could explain why the TFA7 transformant, which only contains two gene cassettes, was able to outperform CelluX™1 and, in some cases, TP1 and AP1 as well. Alternatively, this could also be linked to a lower metabolic burden as compared to TP1 and AP1 (Table 3). In terms of resistance towards cinnamic acids, there was no significant difference between the parental strain and the last set of transformants (FIG. 3A). In contrast, this resistance phenotype was evident in the first round of integration for the PAD1-ICT1 combination (PI3, FIG. 3A) together with increased susceptibility to higher temperatures (FIG. 3C). The reduced thermo-tolerance could be due to an excess fluidity of the membrane caused by a higher proportion of unsaturated fatty acids incorporated by the ICT1p (1-acylglycerol-3-phosphate O-acyltransferase).

The tolerance of the yeast towards lignocellulosic derived inhibitors is also dependent on the carbon source in the fermentation media, and xylose metabolism is much more susceptible than glucose metabolism. However, there is limited information on the interaction of several genes conferring inhibitor tolerance with the XI pathway, especially on industrial strains of S. cerevisiae. These results clearly demonstrate the interlinkage between carbon metabolism and microbial inhibitor resistance. During fermentations of IC media with xylose as the only carbon source, the conversion of furfural was decreased drastically in all the strains (FIG. 4B). However, this reduction was not as severe in the case of the transformants (2.4-4-fold lower) compared to the parental strain (7.4-fold lower) (FIG. 4B). Invariably, the true test of inhibitor resistance is fermentation ability exhibited with lignocellulose hydrolysates. Fermentations in SSL presented a unique challenge to the strain development as it is both xylose rich and contains microbial inhibitors unique to the paper and pulp production process such as ligno-sulphonates and high concentrations of Ca⁺² or Mg⁺² ions, above the typical compliment of weak acids, furans and phenolics.

In 2% YPD supplemented with 40% v/v SSL, transformants TP1 and AP1 outperformed CelluX™1 (4.8% increment on ethanol yield for API1, 11.84% increment on ethanol yield for TP1), confirming enhanced inhibitor resistance phenotypes (Table 3). Given the poor performances of strains in xylose only fermentations supplemented with IC, it had been expected that the strains would not be able to tolerate SSL well. However, this improvement was observed despite a reduction in the xylose consumption compared to the parental strain, especially for the API1 transformant (Table 3). The 80% v/v SSL YPD media proved too toxic for all the strains, with microbial inhibitors atypical to hydrolysates such as MgO, lignosulphonates and SO₂ hypothesized to contribute high osmotic and inhibitor stress on the developed yeast strains.

Unexpectedly, the ethanol production from xylose in the selected transformants was also reduced when fermentations were carried out with no inhibitors present (Table 3, values for YPX). Nonetheless, the transformants containing the TAL1+FDH1 combination from the first integration (TFA7, TP1) were less influenced with about 10% reduction on xylose consumption compared to an almost 58% reduction in the AP1 transformant.

Select gene combinations were further transferred/transformed into the industrial recombinant CelluX™4 strain (without FPS1 gene deletion) and evaluated for inhibitor resistance phenotypes, resultant fermentation ability and xylose capacity.

Transformants were evaluated in 50% w/w SSL and/or sugarcane hydrolysate fermentations under industrially relevant conditions. The 2^{nd} integration transformants i.e. TAF and TFA transformants exhibited improved inhibitor resistance phenotypes in 50% v/v SSL. Additionally, fermentation of the best 2^{nd} integration transformants, TFA1.4, TFA 3.3 and TAF8.4 exhibited significantly higher ethanol titres, productivity and yield then the parental CelluX™4 strains in 50% v/v sugarcane hydrolysate fermentations under industrially relevant conditions. Overall, the CelluX™4 control strain was significantly outperformed by select transformants in terms of ethanol titre, ethanol yield, volumetric productivity, xylose consumption and metabolic yield. The TFA 3.3 transformant produced >40 g/L ethanol in harsh toxic 2G substrate, which is a significant step toward economical 2G bioethanol production.

The CelluX™1 transformants exhibited a reduction in xylose capacity. The CelluX™4 strain development circumvented this, by using screening methods with xylose as the predominate carbon source. The TFA3.3, C4TP3.8, TFA1.4 and C4TP1.3 transformants exhibited high fermentation ability and were further analysed for xylose ability. The transformants exhibited higher initial weight loss, i.e. xylose capacity, during fermentation. However, most transformants exhibited the same weight loss as the CelluX™4 control strain at 24 h. The C4TP3.8 exceeded the control strain weight loss, indicating that this strain may have improved xylose ability above that of the control strain. The use of xylose-rich medium for screening of initial transformants may have allowed for the selection of transformants with improved inhibitor resistance without negatively impacting on xylose ability. This circumvents the loss of xylose ability seen in the earlier CelluX™1 strain development.

The integration of TAL1+FDH1 followed by ARI1+ADH6 (and optionally PAD1+ICT1) resulted in strains with improved tolerance towards furans and formic acid. Selected transformants could outperform the parental strain when grown on SC media supplemented with 40% (v/v) xylose-rich SSL hydrolysate. Furthermore, in undetoxified whole slurry hydrolysates, the transformant strain TP1 demonstrated high acetic acid resistance and significant furan aldehyde and phenolic aromatic aldehyde detoxification phenotypes to achieve high ethanol yields. Selected transformants were screened in 50% w/w sugarcane hydrolysate fermentations under industrially relevant conditions and significantly outperformed the parental strain. The TFA3.3 and TFA1.4 transformants demonstrated high final ethanol titers at 40.1 g/L and 39.4 g/L ethanol. Also, the integration of TAL1 ADH6 followed by ARI1+FDH1 (and optionally PAD1+ICT1) resulted in strains with improved tolerance towards furans and formic acid. The TAF8.4 transformant demonstrated high acetic acid resistance and significant furan aldehyde and phenolic aromatic aldehyde detoxification phenotypes to achieve high ethanol yields, significantly outperforming parental strain.

The invention will now be described in further detail with reference to the following non-limiting examples.

### EXAMPLES

Materials and Methods

1. S. cerevisiae CelluX™1 Strain Development and Fermentation Screening

Microbial Strains and Culture Conditions

S. cerevisiae CelluX™1 (LEAF Technologies, France) was selected as a background strain for rational yeast engineering. S. cerevisiae CelluX™1 and transformants were routinely cultivated, selected and screened using YPD (20 g L⁻¹ glucose, 10 g L⁻¹ yeast extract and 20 g L⁻¹ peptone; Merck, Darmstadt, Germany) media supplemented with 300-400 μg mL⁻¹ of the appropriate antibiotics or combination of antibiotics, namely Hygromycin B (Calbiochem, San Diego, USA), Geneticin and Zeocin (Melford Laboratories Ltd., Ipswich, UK). Strains were pre-cultured in synthetic complete (SC-X) media at pH 5 containing 20 g L⁻¹ glucose and 20 g L⁻¹ xylose, 5 g L⁻¹ (NH₄)₂SO₄, 1.67 g L⁻¹ YNB w/o aa, 3 g L⁻¹ KH₂PO₄ and 100 mM potassium phthalate, supplemented with 20% inhibitor cocktail (20%-IC) containing 0.2 g L⁻¹ cinnamic acid, 0.1 g L⁻¹ HMF, 1.5 g L⁻¹ furfural, 1.2 g L⁻¹ acetic acid and 0.16 g L⁻¹ formic acid (Sigma Aldrich, St. Louis, USA). Pre-cultures were incubated at 30° C. and shaking at 200 rpm. Growth curves of select strains were conducted in YPD and YPDX (20 g L⁻¹ glucose, 10 g L⁻¹ yeast extract, 20 g L⁻¹ peptone, 20 g L⁻¹ xylose; Merck, Darmstadt, Germany), incubated at 30° C., shaking at 200 rpm and sampled at 3 h intervals for 24 h. Escherichia coli DH5a (Life Technologies, CA, USA) was used for plasmid propagation and cloning. E. coli transformants were cultivated at 37° C. in Luria Bertani (LB) media (1% tryptone, 0.5% yeast extract, 1% NaCl; Merck, Darmstadt, Germany) supplemented with 100 μg mL⁻¹ ampicillin (Roche, Johannesburg, South Africa).

Construction of Plasmids

Standard protocols for DNA manipulation were followed. Genomic DNA was extracted from S. cerevisiae BY4742ΔFPS1 and used as template DNA for amplification of the target gene open reading frame. Target genes ARI1, ADH6, FDH1, ICT1, PAD1, and TAL1 were amplified via PCR using the Phusion® high fidelity DNA polymerase (New England Biolabs, Ipswich, USA) and appropriate primers on an Applied Biosystems 2720 thermocycler (Life Technologies, CA, USA) according to the manufacturers' recommendations. Primers introduced Pad and Asci restriction sites required for directional cloning into the delta integration plasmids, pBZD, pBKD and pBHD. PCR products were initially ligated into pCLoneJET 1.2 commercial vector (Thermo Scientific, Waltham, USA) according to the manufacturers' guidelines. Gene sequences were verified using the dideoxy chain termination method and an ABI PRISM™ 3100 genetic analyser (Applied Biosystems, Waltham, USA).

The first gene expression cassettes were constructed by directional cloning of ARI1, TAL1 or PAD1 genes into pBKD1, pBHD1 or pBZD1 (D1) plasmids, respectively, containing the constitutive PGK1 gene promoter and terminator sequences. Secondary gene expression cassettes were constructed by directional cloning of ADH6, FDH1 or ICT1 genes into pBKD2 (D2) plasmid containing the constitutive ENO1 gene promoter and terminator sequences. Double gene expression cassettes were generated by sub-cloning the pBKD2 ENO1ₚₜ gene cassettes as a SpeI/NotI fragment into corresponding pB(K/H/Z)D1 plasmids to yield single delta plasmids with both PGKₚₜ and ENOₚₜ expression cassettes. All key plasmids used and constructed are listed in table 1.

TABLE 1
Final plasmids and yeast strains

| Plasmids/Strain | Relevant genotype |
|---|---|
| pBKD1 | bla δ-site PGK1_{P}-PGK1_{T} kanMX δ-site |
| pBKD-AA | bla δ-site PGK1_{P}-ARI1-PGK1_{T} kanMX ENO1_{P}-ADH6-ENO1_{T} δ-site |
| pBHD1 | bla δ-site PGK1_{P}-PGK1_{T} hphNT δ-site |
| pBHD-TF | bla δ-site PGK1_{P}-TAL1-PGK1_{T} hphNT ENO1_{P}-FDH1-ENO1_{T} δ-site |
| pBZD1 | bla δ-site PGK1_{P}-PGK1_{T} ShBle δ-site |
| pBZD-PI | bla δ-site PGK1_{P}-PAD1-PGKl_{T} ShBle ENO1_{P}-ICT1-ENO1_{T} δ-site |

S. cerevisiae strains

| | |
|---|---|
| S. cerevisiae | BUD5::ADH1_{P}-XKS1-CYC1_{T} TAL1::PGK1p-TAL1-CYC1_{T} TKLI::TDH3ₚ-TKL1- |
| CelluX ™ 1/4 | CYC1_{T} RPE1::TDH3_{P}-RPE1-CYC1_{T} RKI1::TDH3_{P}-RKI1-CYC1_{T} HO::PsXYLZ- |
| | HYGRO BUD5::CpXI-BLAST GRE3/ΔGRE3 |
| S. cerevisiae | CelluX ™ 1 FPS1/ΔFPS1 PGK1_{P}-ARI1-PGK1_{T} kanMX ENO1_{P}-ADH6-ENO1_{T} |
| AA6 | |
| S. cerevisiae | CelluX™ 1 FPS1/ΔFPS1 PGK1_{P}-TAL1-PGK1_{T} hphNT ENO1_{P}-FDH1-ENO1_{T} |
| TF2 | |
| S. cerevisiae PI3 | CelluX™ 1 FPS1/ΔFPS1 PGK1_{P}-PAD1-PGK1_{T} ShBle ENO1_{P}-ICT1-ENO1_{T} |
| S. cerevisiae | CelluX™ 1 FPS1/ΔFPS1 PGK1_{P}-TAL1-PGK1_{T} hphNT_{P}-FDH1-ENO1_{T} PGK1_{P}- |
| TFA7 | ARI1-PGK1_{T} kanMX ENO1_{P}-ADH6-ENO1_{T} |
| S. cerevisiae | TFA7 PGK1_{P}-PAD1-PGK1_{T} ShBle ENO1_{P}-ICT1-ENO1_{T} |
| TP1 | |
| S. cerevisiae | AA6 PGK1_{P}-TAL1-PGK1_{T} hphNT ENO1_{P}-FDH1-ENO1_{T} PGK1_{P}-PAD1-PGK1_{T} ShBle |
| AP1 | ENO1_{P}-ICT1-ENO1_{T} |

Yeast Transformation and Screening

FPS Deletion Stains

The FPS1 gene was disrupted to generate FPS1 gene deletion strains using plasmid pYFCUP1. Plasmid DNA was propagated and extracted using cetyltrimethylammonium bromide (CTAB) plasmid extraction protocol, and used as template to PCR the FPS1L-CUP1-FPS1R insert using Phusion® high fidelity DNA polymerase (New England Biolabs, Ipswich, USA) and appropriate primers. The 3030 bp linear PCR product was separated on 1% agarose gel to confirm the insert. The PCR product was then purified using GeneJet PCR purification kit (Thermo Scientific, Waltham, USA) and transformed into S. cerevisiae CelluX™1 by electroporation using a Bio-Rad Gene-Pulser Apparatus (1.4 kV, 200 OHMS, and 25 μF). Transformants were incubated in 2% YPDS (20 g L⁻¹ glucose, 10 g L⁻¹ yeast extract, 20 g L⁻¹ peptone, 1 M sorbitol) media at 30° C. for 4-5 h, plated on YPDS agar plates supplemented with 7 mM and 8 mM CuSO₄ and incubated for 72 h at 30° C. Transformants were confirmed with PCR using FPS1-L forward and CUP1-L reverse primers. Partial FPS1 gene deletion was confirmed and attributed to polyploidy nature of parental strain. The partial FPS1 gene deletion strains were screened in 70 mL fermentations using 2% YPDX supplemented with sugarcane pretreatment liquor to a concentration of 50% v/v. Fermentations were sampled at 24 h intervals over a period of 7 days and samples were analysed by HPLC for fermentations products (described below).

Strain Construction

The first round of strain construction involved the integration of nine distinct double gene expression cassettes into the partial FPS1 gene deletion CelluX™1 strain. All integration plasmids were digested with either Bst11071 or XhoI (Thermo Scientific, Waltham, USA) according to the manufacturer recommendations and transformed into partial FPS1 gene deletion CelluX™1 strain by electroporation using a Bio-Rad Gene-Pulser Apparatus (1.4 kV, 200 OHMS, and 25 μF). Transformants were recovered on 2% YPD supplemented with appropriate antibiotics and confirmed via PCR using PGKseq-L (D1) and BKDENOpt-L (D2) as forward primers in conjunction with gene-specific reverse primers to confirm complete double gene insert. The transformation frequency of the delta plasmids allowed for a range of copies to be integrated—thus preliminary plate screenings were conducted to identify transformants with higher copy numbers. The plate assays are based on antibiotic resistance, i.e. higher copy numbers transformants exhibit increased antibiotic resistance. The screening was done on 2% YPD plates supplemented with increasing concentrations of appropriate antibiotics. Ten transformants were selected and underwent high throughput screening to determine growth and ethanol yields. Transformants were pre-cultured in 2% YPD supplemented with 20% v/v sugarcane pretreatment liquor for 24 h and inoculated to an OD₆₀₀ of 1 into 5 mL media composed of 2% YPD supplemented with 65% v/v sugarcane pretreatment liquor. Fermentations were conducted in capped 15 mL conical tubes incubated at 30° C., on a rotary wheel at 100 rpm, for 120 h with end-point sampling for fermentation products and growth (0D600). S. cerevisiae CelluX™1 was used as parental control in all screening fermentations.

The second round of integration involved the integration of pBKD-AA and pBHD-TF plasmids into TF2 and AA6 transformants respectively thus generating strains with AA and TF double gene expression cassettes in different combinations. Plasmids were linearized with XhoI (Thermo Scientific, Waltham, USA) and transformed into selected strains via electroporation. Transformants were recovered on 2% YPD plates supplemented with appropriate antibiotics and confirmed via PCR using appropriate primer combinations. Twenty-four confirmed transformants were selected for each combination (TF2+pBKD-AA or AA6+pBHD-TF) and screened in 2% YPD supplemented with 65% v/v pretreatment liquor.

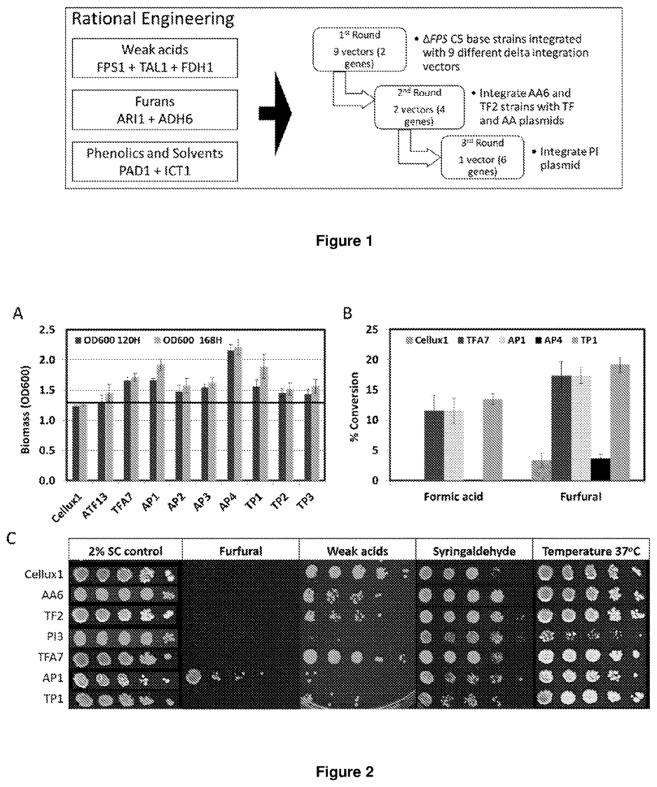

The final integration involved the addition of pBZD-PI expression cassette to the second round combinations to generate TF2-pBKD-AA+pBZD-PI and AA6-pBHD-TF+pBZD-PI overexpression strains. Transformation efficiency decreased after each round of sequential integration, with only six strains per combination recovered and confirmed via PCR after the third round of integration. Strains were screened in 2% SC-X supplemented with 65% v/v pretreatment liquor and spiked with 20 g L⁻¹ furfural and formic acid, and the final strains selected overexpressed six inhibitor resistance genes in different combinations. Strain names were derived from the initials of the genes inserted, e.g. TF is TAL1+FDH1. For the second round transformants, the initial of the second integration (D1 gene) was added to initials of the first integration, e.g. TF+ARI1 resulting in TFA. For the third round transformants, the initial of the first integration D1 gene and initial of the third D1 gene was combined, e.g. TAL1 (D1 gene first integration)+PAD1 (D1 gene third integration)=TP. The constructed strains are shown in FIG. 1.

Fermentations with Single Inhibitor Group and Inhibitor Cocktail

Fermentations were conducted in 2% SC-X minimal media supplemented with either: 1 g L⁻¹ cinnamic acid at pH 5.0, 5 g L⁻¹ furfural and 0.5 g L⁻¹ HMF at pH 5.0, or 5 g L⁻¹ acetic acid and 0.81 g L⁻¹ formic acid at pH 5.0. Fermentations in 2% SC-X supplemented with inhibitor cocktail (IC) contained 6 g L⁻¹ acetic acid, 0.8 g L⁻¹ formic acid, 5 g L⁻¹ furfural, 0.5 g L⁻¹ HMF and 0.5 g L⁻¹ cinnamic acid. The concentration of cinnamic acid at 0.5 g L⁻¹ was selected to account for total phenolic content of spent sulphite liquor (SSL). Strains were pre-cultured in 50 mL 20%-IC SC-X media pH 5.0 until late exponential/early stationary phase and inoculated into 50 mL 2% SC-X or 2% SCX (xylose only) media supplemented with appropriate inhibitors, to an OD₆₀₀ of 1. Fermentations were incubated at 30° C., shaking at 200 rpm for 120 h, with sampling at 24 hour intervals. Fermentations were inoculated in triplicate with S. cerevisiae CelluX™1 as industrial and parental control strain. Media controls were included in all fermentations to account for the evaporation of volatile inhibitor compounds. The inhibitor cocktail composition is based in part on inhibitor concentrations found in both SSL and sugarcane steam explosion liquor. Synthetic media supplemented with inhibitors were selected to exercise better control over experimental parameters given the inherent unknown/unquantifiable mix of inhibitors present in pretreatment liquor/SSL.

Fermentations with Undetoxified Hydrolysates

Strain performance in industrially relevant hydrolysates was evaluated in concentrated SSL and steam explosion and undetoxified sugarcane bagasse (SCB) whole slurry hydrolysates.

SSL Fermentation

Fermentations with concentrated SSL was conducted to ascertain strain performance under industrially relevant fermentation conditions. SSL was provided by Sappi Saiccor (Umkomaas, South Africa), which uses an acid-based sulphite pulping process. Strains were pre-cultured in 2% SC-X media supplemented with 20% v/v SSL to late exponential/early stationary phase (0D600>10) and inoculated into 100 mL serum bottles with 50 mL fermentation media to an OD₆₀₀ of 1 (˜0.6 g L⁻¹ DW). Fermentation media consisted of 2% SC media (20 g L⁻¹ glucose, 5 g L⁻¹ (NH₄)₂SO₄, 1.67 g L⁻¹ YNB w/o aa, 3 g L⁻¹ KH₂PO₄ and 100 mM potassium phthalate), supplemented with either 40% (pH 5) or 80% (pH 4.5) v/v concentrated SSL. Samples were taken every 24 h and analysed via HPLC.

Preparation of Steam Explosion Pretreatment Liquor

Industrial sugarcane bagasse (SCB) was collected from two local sugar mills in Malelane (Mpumalanga, South Africa) and Mount Edgecombe (Kwazulu Natal, South Africa) and prepared as previously described. Steam explosion (StEx) of SCB was conducted in an automated batch pilot-scale unit (IAP GmBH, Graz, Austria) equipped with a 19 L StEx reaction vessel, a 100 L discharge vessel and a 40-bar steam generator using a protocol and conditions previously described. The resultant pretreated slurry was separated into a solid and liquid phase (liquor) using a pneumatic press (Nesco Engineering, Cape Town, South Africa) and the pretreatment liquor was collected, aliquot and refrigerated at −20° C. until use.

Preparation and Fermentation of Steam Exploded SCB Whole Slurry Hydrolysate

In preparation of the whole slurry hydrolysate, SCB was steam exploded at 200° C. for 10 min using the aforementioned pilot-scale reactor. The StEx-SCB whole slurry was enzymatically hydrolysed in a 100 mL baffled Erlenmeyer flask at 15% (w/w) solids loading, and an enzyme dosage of 40 mg protein per gram glucan. Commercial fungal enzyme preparations Cellic® CTec2 and Cellic® HTec2, donated by Novozymes (Copenhagen, Denmark), were used as the hydrolytic enzyme cocktail at a previously optimized mixture of 85% Cellic® CTec2 and 15% Cellic®HTec2 on a weight basis. The enzymatic hydrolysis mixtures were supplemented with 50 mM phosphate buffer and 50 mg L⁻¹ chloramphenicol to maintain the hydrolysis pH and prevent bacterial contamination, respectively, and subsequently incubated at 50° C. and 180 rpm in an orbital shaker. After 72 h of hydrolysis, the hydrolysis slurry was centrifuged at 8,000×g for 30 min. The sugar-rich supernatant was supplemented with 0.5% (w/w) corn steep liquor, pH adjusted to 5.5 using 10 M KOH, before being filter sterilized through a 0.22 μm PES filter into a sterile bottle. The sterile whole slurry hydrolysate was refrigerated at 4° C. and used within 24 hours.

Whole slurry hydrolysate fermentation was evaluated using the transformant strain TP-1, with the fourth generation of CelluX™1, viz. CelluX™4, used as the industrial control strain. Pre-seed cultures of TP-1 and CelluX™4 were pre-cultivated from glycerol stock cultures in test tubes containing 10 mL 2% SC media and incubated at 30° C. and 150 rpm overnight. Seed cultures were prepared by inoculating the pre-cultures into pre-conditioning media consisting of 75 mL of YPDX media (75 g L⁻¹ glucose, 25 g L⁻¹ xylose, 10 g L⁻¹ yeast extract, 20 g L⁻¹ tryptone) and 25 mL steam explosion pretreatment liquor to an OD₆₀₀ of 1. After a 18 h incubation period, the primed seed-cultures were harvested by centrifugation at 1,500×g for 10 min and the yeast pellets inoculated into 100 mL serum bottles with 50 mL undetoxified whole slurry hydrolysate to an OD₆₀₀ of 3 (˜1.8 g L⁻¹ DW).

Calculations and Statistical Analysis

All experiments were conducted in triplicate. Data were analysed using Microsoft Excel data analysis tools whereby triplicate values were subjected to analysis of variance (ANOVA) and p-value less than 0.05 was considered significant. Ethanol yields (Y_{E/TS}) were calculated as final ethanol g L⁻¹ divided by total sugars g L⁻¹ at t=72 h, while ethanol productivity was calculated as ethanol concentration g/L divided by fermentation time (h). The metabolic yield was calculated as final ethanol g L⁻¹ relative to the stoichiometric maximum ethanol from the consumed glucose and xylose. The growth rate as μMax was determined during the exponential growth phase by plotting the natural logarithm values as a function of time. The specific glucose or xylose uptake (q_{glucose} or q_{xylose}) and specific ethanol production rates (qₑₜₕₐₙₒₗ) were determined from the amount of substrate consumed or ethanol produced per cell mass.

2. S. cerevisiae CelluX™4 Strain Development and Fermentation Screening

Strain Development

CelluX™4 was transformed using TA (TAL1+ADH6), AF (ARI1+FDH1), AA (ARI1+ADH6), TF (TAL1+FDH1) and PI (PAD1+ICT1) delta integration cassettes. The first cassette integrated was either the TA or TF cassette. All transformants were then screened for fermentation efficiency in 50% v/v SSL. The two best TA and TF transformants were then selected and transformed with the AF and AA cassettes, respectively. The two best TFA transformants were then transformed with the PI delta integration cassette, Transformants were selected using appropriate antibiotic selection and confirmed using PCR. Transformants were named based on the cassette integration sequence. For example, transformants with TF+AA were classified as “TFA transformants” and transformants with TA+AF combination were classified as TAF transformants. The final strains are designated C4TP1 and C4TP3, i.e. CelluX™4+TAL1+FDH1+ARI1+ADH6+PAD1+ICT1.

SSL Fermentation Set-Up

Fermentations were conducted in 250 mL Balloon flasks, containing 50 mL of fermentation medium, as SSL quantity was limited. Transformants were preconditioned in YPX (20 g/L peptone, 20 g/L xylose, 10 g/L yeast extract) supplemented with 20% v/v SSL, and incubated at 30° C., shaking at 120 rpm for 2-3 days to reach OD>50. The fermentation medium consisted of 20 g/L peptone, 10 g/L yeast extract and 50% v/v SSL as carbon source, with 0.2 g/L chloramphenicol to control for contamination. The medium was pH adjusted with KOH to pH ˜5.2 with no added buffer capacity. The fermentations were inoculated to an OD of 3.0 and continued for 120 h, at 30° C., shaking at 120 rpm with weight loss taken every 24 h. Endpoint sampling was conducted to analyze for ethanol, glycerol, xylose, glucose, acetic acid and lactic acid. The initial xylose and glucose content was ˜40-50 g/L and 5-10 g/L, respectively. All fermentations were conducted in triplicate.

Sugarcane Bagasse Hydrolysate Fermentations

The C4TP1 and C4TP3 transformants were screened in 50% w/w sugarcane bagasse hydrolysate. Transformants were initially cultured in 10 mL 2% YPD (20 g/L Glucose, 20 g/L Peptone, 10 g/L Yeast extract) at 30° C., shaking at 120 rpm, overnight. Cultures were further propagating in 100 mL 2% YPD for an additional 24 h. Cultures were harvested at 4000 rpm for 5 minutes, resuspended in PBS buffer at pH 5.0. Dry weight of cultures were determined and fermentations were then inoculated to 0.5 g DCW/kg of the medium. Fermentations were conducted in 250 mL balloon flasks, containing 100 g fermentation medium consisting of 58 g/kg glucose, 21 g/kg xylose, 0.01 g/kg riboflavin, 0.2 mg/kg biotin, 5 g/kg urea, 0.93 g/kg orto-phosphoric acid, 0.2 g kg chloramphenicol, 5 g/kg vitamin solution (25 g/kg inositol, 2 g/kg thiamine, 2 g/kg pyridoxine, 2 g/kg nicotinic acid, and 0.4 g/kg 4 aminobenzoic acid) and 2 g/kg mineral solution (370 g/kg MgSO₄.7H₂O, 0.15 g/kg CuSO₄.5H₂O, 0.3 g MnCl₂.4H₂O, ZnSO₄.4H₂O). Fermentation pH was adjusted to 5.2 using KOH. Fermentations continued for 88 h, at 30° C., shaking at 120 rpm with weight loss taken ×3 times per day. Endpoint sampling was conducted to analyze for ethanol, glycerol, xylose, glucose, acetic acid and lactic acid.

Xylose Phenotype Characterization

As the xylose utilization phenotype was negatively impacted in the previous examples, the xylose phenotype of the best fermenting 2^{nd} integration transformants (TFA1.4, and TFA3.3) and 3^{rd} integration transformants (C4TP1.3 and C4TP3.8) was determined under non-selective, rich medium conditions, with CelluX™4 as control strain. The xylose phenotype was determined using 2% YPX (20 g/L xylose, 20 g/L peptone, 10 g/L yeast extract). Cultures were prepared in 5 mL 2% YPD, incubated overnight at 30° C. on rotor wheel, and used to inoculate 100 mL 2% YPX medium in 250 mL balloon flasks to an OD₆₀₀ (optical density) of 0.5. The cultures where then incubated at 30° C., shaking at 120 rpm for 24 h. Weight loss was recorded every 2 h for the initial 14 h, and final sampling at 24 h, with weight loss as indicator of active xylose fermentation. All fermentations were done in triplicate.

Results

1. Transformed CelluX™1 Strains

Strain Development in Lignocellulose Pretreatment Liquor and Inhibitor Tolerance Assays

The strain construction strategy centred on three rounds of sequential delta integration of double gene expression cassettes to construct strains with three double gene expression cassettes, i.e. strains overexpressing selected inhibitor resistance genes in different combinations. Overexpression of respective double gene cassettes was facilitated by homologous recombination of delta integration cassettes into native delta sequences distributed in the parental CelluX™ 1 yeast genome. Transformants were screened for growth and ethanol yield in lignocellulosic retreatment liquor after each round of integration to select base strains for the next round of transformation, with final transformants assayed for inhibitor tolerance phenotypes as well.

Before the integration of inhibitor tolerance genes commenced, a partial FPS1 gene deletion variant of S. cerevisiae CelluX™1 was generated. Eight partial FPS1 gene deletion transformants were selected for by screening for higher ethanol yield (g ethanol g⁻¹ total sugar) on 2% YPD supplemented with 65% v/v sugarcane pretreatment liquor. The best performing transformant CelluX™1ΔFPS1-05 yielded 0.41 g g⁻¹ at 169 h that showed a 5% ethanol yield increment to the parental CelluX™1 strain at 0.39 g g⁻¹. Interestingly, strain CelluX™1ΔFPS1-C5 also exhibited an increase of 19.8% in formic acid detoxification and higher xylose consumption at 53.6% compared to 49.8% of parental strain. This partial FPS1 gene deletion CelluX™1 ΔFPS1-05 strain was used as the host for the first stage integration.

The results from the best transformant per gene combination from the first round of integration are listed in Table 2. The first round of transformants often exhibited a trade-off between growth and ethanol yield. The pBKD-AA integration cassette with the ARI1 and ADH6 genes that confer furan resistance, in combination with partial FPS1 gene deletion, allowed for furan and weak acid resistance. Final ethanol yields of pBKD-AA transformants showed overall improvement that ranged from 0.34-0.38 g g⁻¹ as compared to parental strain at 0.33 g g⁻¹, with a maximum increase in the ethanol yield of 15.8% (Table 2). Partial deletion of the FPS1 gene in combination with pBKD-AA integrations proved beneficial to inhibitor resistance phenotype in terms of cell growth, as 80% of transformants displayed similar or increased growth (measured in absorbance) compared to the parental strain. The pBKD-AF integration cassette overexpressing the ARI1 and FDH1 genes also conferred furan and weak acid resistance. This configuration, however, significantly decreased the ethanol yield by 4.29-17.2%, compared to the parental strain, although an improvement in growth was seen with AF10, showing an increment of 7.92% over the parental strain. The pBKD-AI integration cassette overexpressing the ARI1 and ICT1 genes conferred furan, organic solvent and weak acid resistance. However, 62.5% of transformants with this configuration exhibited decreased growth of 12-15%. Likewise, ethanol yields decreased by 1-7%, showing improvement for only one strain (All) with increments of 1.25% over the parental control, respectively.

TABLE 2
Performance of the best transformant per gene combination after 1st stage of
strain development.

| | | | | Growth |
|---|---|---|---|---|
| Gene | | | EtOH Yield | (OD₆₀₀) % |
| combination | Strain | Resistance phenotype | % increment | increment |
| ARI + ADH6 | AA6 | Furans + Weak acids | 15.8 | 19.5 |
| ARI1 + FDH1 | AF10 | Furans + Weak acids | −5.55 | 7.92 |
| ARI1 + ICT1 | AI1 | Furans + Weak acids + organic solvents | 1.25 | −12 |
| PAD1+ ADH6 | PA7 | Phenolics + Furans + Weak acids | −1.97 | 3.40 |
| PAD1 + FDH1 | PF5 | Phenolics + Weak acids | −15.4 | 31.8 |
| PAD1 + ICT1 | PI3 | Phenolics + Weak acids + organic | −3.58 | 20.9 |
| | | solvents | | |
| TAL1 + ADH6 | TA6 | Furans + Weak acids | 3.60 | 18.6 |
| TAL1 + FDH1 | TF2 | Weak acids | 16.9 | 6.2 |
| TAL1 + ICT1 | TI10 | Weak acids + organic solvents | −8.34 | −22.3 |

The pBZD-PA integration cassette overexpressing the PAD1 and ADH6 genes and this configuration conferred resistance to furans, phenolics and weak acids. Overall, ethanol yields of the transformants were lower by 2-24.6% or equal to that of the parental strain, with six transformants showing improved growth by 1-18.8% growth increment. The pBZD-PF cassette integration overexpresses the PAD1 and FDH1 genes that confers resistance to phenolics and weak acids. These transformants also exhibited no increases in ethanol yields, but showed an increase in growth by 5-31.7% for most of the transformants. A similar trend was observed in the transformants with the pBZD-PI integration cassette overexpressing the PAD1 and ICT1 genes, which confers resistance to weak acids, phenolics and organic solvents. Ethanol yields were either lower or similar to the parental strain, whereas growth was either similar or higher than that of the reference strain, with the PI3 transformant showing a maximum growth increment of 20.9%.

The pBHD-TA integration cassette overexpresses the TAL1 and ADH6 genes that confer furan and weak acid resistance. The pBHD-TA transformants showed no real differences in ethanol yields relative to the parental strain, with only TA6 showing an improvement at 3.6% ethanol yield increment and final growth increment of 18.6%. Transformants with the pBHD-TF integration cassette overexpressing the TAL1 and FDH1 genes conferring weak acid resistance exhibited similar or higher ethanol yields relative to the parental strain, with TF2 showing the highest increment in yield at 16.9%. The pBHD-TI integration cassette overexpressing the TAL1 and ICT1 genes that confers resistance to weak acids and organic solvents proved to be detrimental to both the ethanol yield and growth, as transformants exhibited significant decreases in both ethanol yield (8.4-35.2%) and growth (20.4-38.2%).

At the conclusion of the first stage of integration, the pBHD-TF and pBKD-AA integration cassettes generated transformants with more than 10% increment on ethanol yield relative to the parental strain. Therefore, these combinations were selected to continue into the second stage that involved the integration of the pBHD-TF and pBKD-AA integration cassettes into S. cerevisiae AA6 (resulting in ATF transformants) and TF2 (TFA transformants), respectively. The second round of transformants also resulted in a trade-off between growth and ethanol yield. Compared to the parental strain, 70.8% of the ATF transformants favoured growth, with ATF13 showing an improved growth increment at 17.3%. Conversely, 58.3% of the TFA transformants exhibited increased ethanol yields. Interestingly, when the concentration of pretreatment liquor in YPDX reduced from 65% v/v to 50% v/v, no significant differences were observed in resistance phenotypes between the parental strain and transformants, indicating possible phenotypic plasticity in transformants.

In the third and final stage, pBZD-PI was integrated into the ATF13 (AP transformants) and TFA7 (TP transformants) strains to generate strains that overexpress six genes in the partial FPS1 gene deletion background. Transformants would therefore exhibit resistance to weak acids, furans and phenolic compounds. The strains were evaluated on growth (absorbance), fermentation ability (ethanol yield) and inhibitor detoxification (% conversion). S. cerevisiae CelluX™1 was used as an industrial and parental reference strain, and ATF13 and TFA7 were used as additional parental control strains. All transformants showed a notable improvement in growth compared to the parental CelluX™1 strain during fermentations with 2% SC-X media supplemented with 65% v/v sugarcane pretreatment liquor at pH 5 and spiked with 20 g L⁻¹ of furfural and 20 g L⁻¹ of formic acid (FIG. 2A). The AP and TP transformants growth profiles surpassed that of the industrial CelluX™1 strain. Interestingly, TFA7, with only 2 gene cassettes, did as well as the final transformants. AP1 and AP4 showed the highest growth (OD₆₀₀), whereas TP1 was the best performer from the TP transformants (FIG. 2A). Only AP1 and TP1 showed a significant difference in growth between 120 h versus 168 h. As expected, ethanol concentrations were very low, ranging from 1.6-2.4 g L⁻¹ given the extreme toxicity of fermentation media (data not shown).

The transformants showed a significant difference in inhibitor detoxification phenotypes for formic acid (present as formate in the medium at pH 5) and furfural (FIG. 2B). Although AP4 showed the highest OD₆₀₀ at 168 h, the growth could not be linked back to an improved inhibitor resistance phenotype. In contrast, the TP1 strain showed the highest detoxification phenotype with an average of 13.4% and 19% conversion of formic acid (formate) and furfural respectively at 168 h (FIG. 2B), outperforming the industrial control strain CelluX™ 1. As a control strain, it showed poor growth and poor inhibitor detoxification with no formic acid converted and only 3% of the furfural detoxified. TFA7 and AP1 transformants similarly surpassed the control strain, with notable differences in detoxification phenotypes.

Two different assay methods were applied to evaluate inhibitor tolerance phenotypes i.e. inhibitor tolerance plate assays (pH 4.0-4.5, no pH control) and enzymatic assays. Plate assays showed variations between inhibitor phenotypes within the three stages of strain development (FIG. 2C). In particular, the PI3 strain show an increased susceptibility to weak acid stress (6 g L⁻¹ acetic acid and 0.8 g L⁻¹ formic acid) when no pH control was implemented (pH<5) and this phenotype was confirmed in AP1 and TP1 transformants with pBZD-PI inserts at the third stage (FIG. 2C), although this integration improved resistance to the phenolic syringaldehyde. AP1 was the only transformant showing resistance to 1 g L⁻¹ furfural when critical mass was present. The in vitro activities of detoxification enzymes were assayed to determine inhibitor detoxification potential of transformants. Detoxification was measured as the decrease in substrate, i.e. furfural, cinnamic acid or formic acid, due to enzymatic degradation. No significant differences were observed between transformants and parental control in furfural assays. In the cinnamic acid assays, PI3 and TP1 transformants exhibited enhanced in vivo cinnamic acid detoxification activity. Similarly, formic assays showed AP1 and TP1 transformants to have enhanced formic acid detoxification phenotypes, relative to the control.

Detoxification Phenotypes in Simulated/Synthetic Inhibitor Cocktail Fermentations

The TFA7, AP1 and TP1 transformants were subjected to fermentations in 2% SC-X media supplemented with either 5 g L⁻¹ furfural and 0.5 g L⁻¹ HMF, 6 g L⁻¹ acetic—and 0.81 g L⁻¹ formic acid, or 1 g L⁻¹ cinnamic acid in order to ascertain detoxification phenotypes of the gene combinations to specific microbial inhibitors groups. The S. cerevisiae CelluX™1 strain was used as both an industrial and parental control. The transformants from the first round of integration (AA6, TF2 and PI3) were used as secondary controls to determine if second and third round transformants exhibit phenotype from first integrations, i.e. cumulative phenotypes.

In fermentations with 1 g L⁻¹ cinnamic acid, there were no differences observed between parental CelluX™1 strain and the TFA7 or TP1 transformants. However, the PI3 transformant exhibited an enhanced cinnamic acid detoxification phenotype (FIG. 3A). The AA6, TFA7, AP1 and TP1 transformants showed marked improvement in furfural detoxification phenotype when compared to parental and industrial control strains. All transformants exhibited a decrease in lag phase of 24 h compared to the 48 h for the parental strain, with furfural detoxified within said time period (FIG. 3B/C). At 48 h, transformants exhibited ethanol yields ranging from 0.25-0.29 g g⁻¹, whereas both industrial and parental control fermentations only had yields of 0.08 and 0.07 g g⁻¹, respectively (FIG. 3D). Glucose was depleted within 48 h versus 72 h for the parental control strain with no significant differences in ethanol yield for TFA1 and AA6 strains versus the CelluX™1 control at 72 h. No significant differences in CelluX™1 and TFA7 fermentation performances were observed with weak acid exposure. However, this fermentation confirmed that the AP1 and TP1 strains are more susceptible to weak acids due to pBZD-PI insert (FIG. 3C).

Fermentations with inhibitor cocktail were conducted with 2% SC supplemented with an inhibitor cocktail (IC) based on the composition of SSL. Blank media supplemented with IC were used as references to account for the evaporation of volatiles. Two different carbon sources were used to determine the possible effect carbon source may have on resistance phenotypes, given the sensitivity of the introduced heterologous pathways to fermentation stresses. As anticipated, strains showed a significant difference in observed inhibitor resistance phenotypes in fermentations with glucose and xylose versus xylose only as carbon source (FIG. 4). Transformants in xylose only fermentations showed poor detoxification phenotypes with <5% of inhibitor compounds detoxified. In fermentations with both glucose and xylose, differences in detoxification phenotypes between transformants and the CelluX™1 control were observed. AP1 outperformed CelluX™1 for furfural detoxification, whereas the TP1 transformant outperformed both AP1 and CelluX™1 for formic acid detoxification. Overall, the transformants exhibited enhanced detoxification phenotypes compared to the CelluX™1 parent.

Lignocellulose Fermentations and Final Growth Kinetics of Transformants

SSL Fermentation

Various concentrations of untreated SSL were used to characterise strain performances in lignocellulose fermentations in terms of consumption percentage of glucose and xylose, ethanol concentration, ethanol yield and ethanol productivity (Table 3). In 2% SC media supplemented with 40% v/v concentrated SSL at pH 5.0, glucose was depleted within 72 h. However, xylose consumption was less than 10% for all strains with the AP1 transformant showing no xylose consumption. Ethanol yields at 72 h showed that the TP1 transformant with a yield of 0.255 g g⁻¹ performed better than the CelluX™1 strain with yields of 0.228 g g⁻¹ (Table 3), an 11.8% increment in yield above the parental control. In 2% SC supplemented with 80% v/v concentrated SSL at pH 4.5, both the parental and transformant strains showed no growth. However, the strains appeared to be metabolically active, as seen by the % consumption of sugars (Table 3). Glucose consumption for all strains exceeded 10%, with CelluX™1, TFA7 and TP1 consuming 15.1%, 15.8% and 16.3%, respectively. However, ethanol concentrations remained below 1 g L⁻¹, with only the CelluX™1, TFA7 and TP1 strains producing ethanol, at 0.51, 0.66 and 0.48 g L⁻¹, respectively.

The growth kinetics of the final transformants were characterised in 2% YPDX and YPX at pH 5.0 in terms of consumption percentage of glucose and xylose, ethanol concentration, ethanol yield, ethanol productivity, metabolic yield and μMax (Table 3). In 2% YPDX, the TFA7 and TP1 transformants exhibited both increased ethanol yield and productivity at 0.428 g g⁻¹ and 0.713 g L⁻¹ h⁻¹ and 0.432 g g⁻¹ and 0.720 g L⁻¹ h⁻¹, respectively. The co-fermentation of glucose and xylose was only reduced for strain API1 during YPDX fermentations, unlike the low xylose utilisation for all strains seen in SSL, highlighting the pronounced effect of inhibitors on % xylose consumption. Furthermore, xylose consumption in 2% YPX decreased from 100% of parental CelluX™1, to 42.3-90.1% for transformants, indicating that the strain modification impacted negatively on xylose capacity. This was confirmed in SSL fermentations with xylose as the main carbon source, where transformants exhibited lower % xylose consumption versus parent CelluX™1.

TABLE 3
Fermentation kinetic parameters of recombinant S. cerevisiae strains and
control strains.

| | | | | | | | | |
|---|---|---|---|---|---|---|---|---|
| | Glucose | Xylose | | Ethanol | | Metabolic | Media | μ_{Max} |
| Strain | cons. % | cons. % | g L⁻¹ | Y_{P/S} g g⁻¹ | g L⁻¹ h⁻¹ | yield % | pH | (h⁻¹) |

YPDX_{t=24 h}

| | | | | | | | | |
|---|---|---|---|---|---|---|---|---|
| CelluX ™1 | 100 | 100 | 17.0 ± 0.13 | 0.424 ± 0.00 | 0.707 ± 0.01 | 82.8 | 5 | 0.572 |
| TFA7 | 100 | 100 | 17.1 ± 0.13 | 0.428 ± 0.00 | 0.713 ± 0.01 | 83.5 | 5 | 0.557 |
| AP1 | 100 | 45.7 | 12.2 ± 0.09 | 0.305 ± 0.00 | 0.508 ± 0.00 | 82.1 | 5 | 0.513 |
| TP1 | 100 | 100 | 17.3 ± 0.08 | 0.432 ± 0.00 | 0.720 ± 0.00 | 84.4 | 5 | 0.545 |

YPX_{t=24 h}

| | | | | | | | | |
|---|---|---|---|---|---|---|---|---|
| CelluX ™1 | — | 100 | 8.18 ± 0.05 | 0.396 ± 0.02 | 0.341 ± 0.00 | 77.7 | 5 | 0.412 |
| TFA7 | — | 90.1 | 7.52 ± 0.25 | 0.364 ± 0.01 | 0.313 ± 0.01 | 78.3 | 5 | 0.364 |
| AP1 | — | 42.3 | 3.81 ± 0.02 | 0.184 ± 0.00 | 0.159 ± 0.00 | 73.7 | 5 | 0.442 |
| TP1 | — | 88.5 | 7.47 ± 0.35 | 0.362 ± 0.02 | 0.311 ± 0.01 | 78.9 | 5 | 0.372 |

40% v/v SSL_{t=72 h}

| | | | | | | | | |
|---|---|---|---|---|---|---|---|---|
| CelluX ™1 | 100 | 5.8 | 11.6 ± 0.93 | 0.228 ± 0.00 | 0.161 ± 0.01 | — | 5 | — |
| TFA7 | 100 | 3.0 | 11.7 ± 0.71 | 0.225 ± 0.01 | 0.162 ± 0.01 | — | 5 | — |
| AP1 | 100 | 0 | 11.8 ± 1.06 | 0.239 ± 0.00 | 0.160 ± 0.02 | — | 5 | — |
| TP1 | 100 | 1.28 | 12.2 ± 1.17 | 0.255 ± 0.03 | 0.169 ± 0.02 | — | 5 | — |

80% v/v SSL_{t=241 h}

| | | | | | | | | |
|---|---|---|---|---|---|---|---|---|
| CelluX ™1 | 15.1 | 1.74 | 0.51 ± 0.03 | n.d.* | — | — | 4.5 | — |
| TFA7 | 15.8 | 1.88 | 0.66 ± 0.03 | n.d.* | — | — | 4.5 | — |
| AP1 | 10.6 | 0.76 | n.d.* | — | — | — | 4.5 | — |
| TP1 | 16.3 | 1.55 | 0.48 ± 0.05 | n.d.* | — | — | 4.5 | — |

*Not detected/determined

Whole Slurry Hydrolysate Fermentation

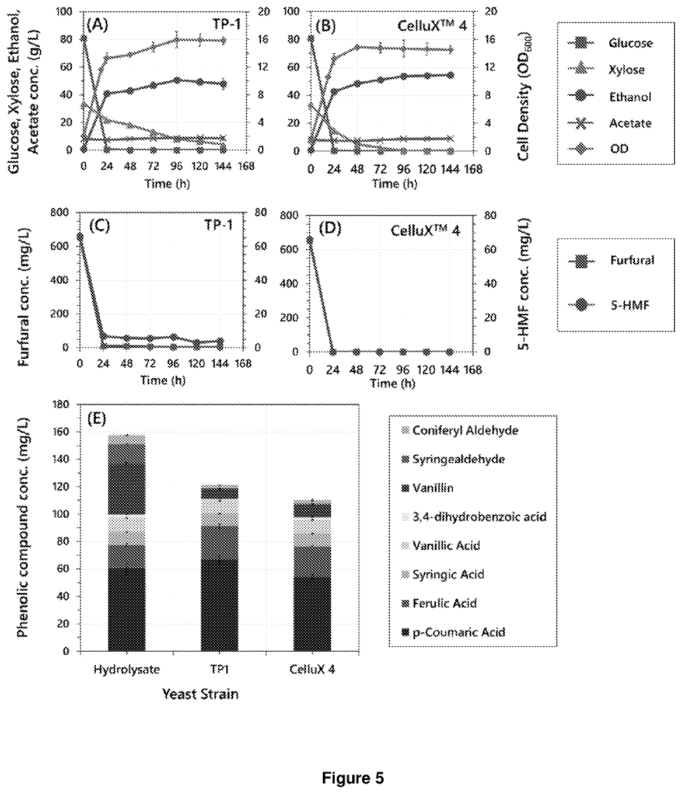

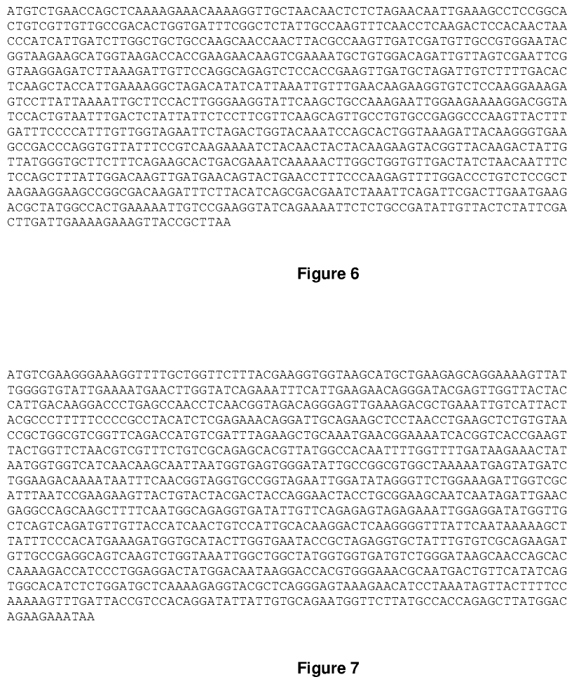

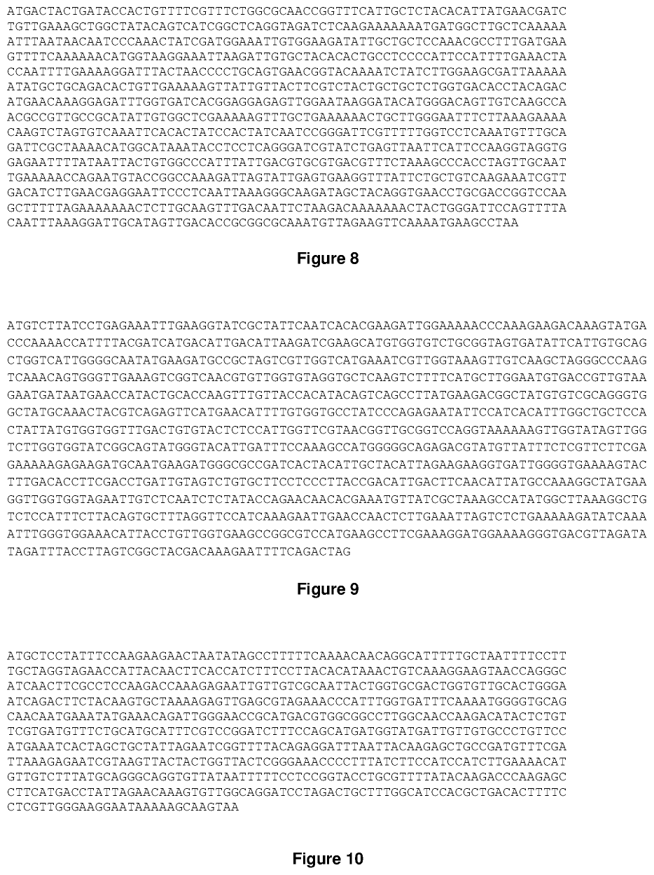

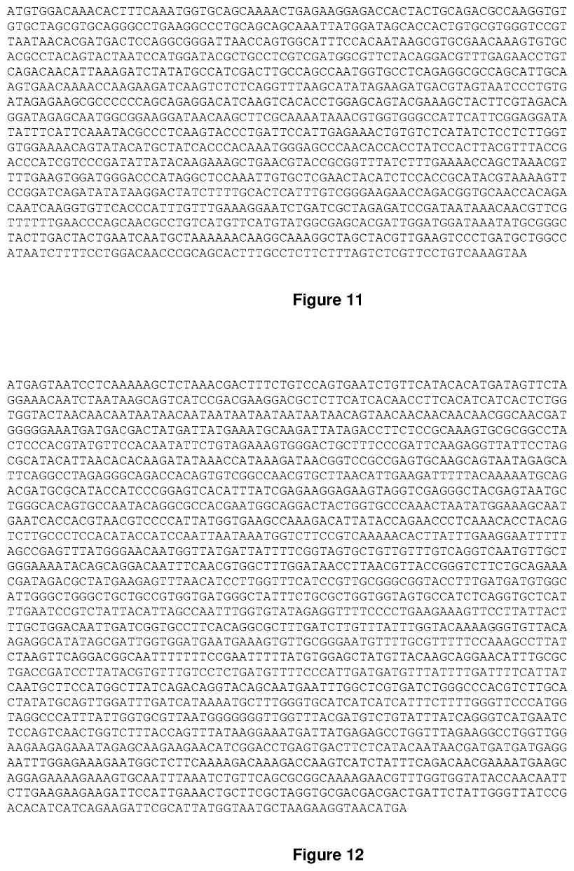

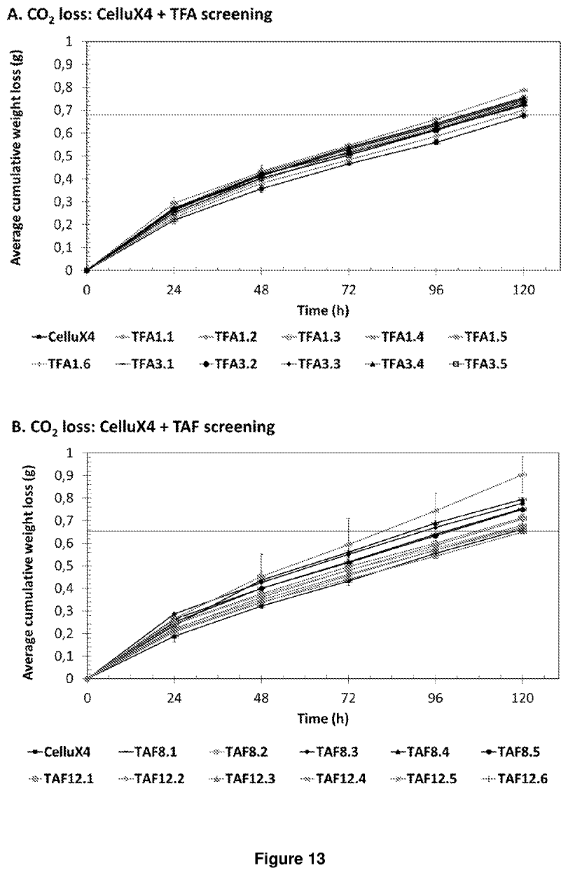

The fermentation capacity of the transformant strain TP1 was evaluated on StEx-treated and undetoxified SCB whole slurry hydrolysate and benchmarked against the performance of the industrial strain CelluX™4. The undetoxified hydrolysate was generated from high solids loading enzymatic hydrolysis to attain high initial sugar concentrations to simulate the synergistic action of multiple stress conditions (including inhibitors and osmotic stress) at the beginning of the fermentation and high ethanol stress on xylose-utilization towards the end of the fermentation. As demonstrated in FIG. 5 (a, b), both TP-1 and CelluX™4 demonstrated rapid glucose consumption within 24 h in the inhibitor-laden whole slurry hydrolysate without a noticeable lag phase. Furthermore, both strains demonstrated near-complete furan aldehyde detoxification phenotypes within 24 h (FIG. 5 (c, d)), suggesting that the assimilation of the furan aldehydes during the glucose consumption phase mitigated their effect on the xylose fermentation capacity of both TP-1 and CelluX™4. After a 144 h fermentation period, the transformant TP1 strain consumed 88% of the xylose in the whole slurry hydrolysate, resulting in a final ethanol concentration of 50 g L⁻¹, an ethanol yield of 0.436 g g⁻¹, and a metabolic yield of 89%. In comparison, CelluX™4 achieved near-complete xylose fermentation within 72 h, demonstrating a higher ethanol concentration (53.8 g L⁻¹), ethanol yield (0.467 g g⁻¹) and volumetric ethanol productivity (0.747 g L⁻¹ h⁻¹) relative to the transformant strain TP-1. In both cases, no xylitol production was detected after 144 h.

Quantification and comparison of the phenolic aldehydes and phenolic acid concentrations in the hydrolysate before and after fermentation revealed that the lignin-derived aromatic aldehydes, viz. vanillin, syringaldehyde, and coniferyl aldehyde, were present at significantly lower concentrations after the fermentation relative to the initial hydrolysate (P<0.05). Although they are typically present at significantly lower concentrations relative to furan aldehydes and aliphatic acids, phenolic aldehydes such as coniferyl aldehyde and vanillin have previously demonstrated significantly higher S. cerevisiae toxicity, even at low concentrations. The phenolic aromatic acids remained largely unchanged, except for p-coumaric acid, which was reduced by 10% during the whole slurry hydrolysate fermentation with CelluX™4.

Direct comparison of the fermentation of undetoxified hydrolysates of various strains presented in literature is not trivial due to the differences in the source of raw biomass, type of pretreatment, pretreatment conditions, background of the selected strains, and ultimately the levels multiple stress factors (e.g. ethanol, salt content, inhibitors) present in the hydrolysates. Among the most efficient xylose fermenting yeasts reported in literature, recombinant S. cerevisiae strains RWB218, GS1.11-26, XH7, and LF1 have demonstrated high xylose consumption (>80%), ethanol concentrations (>38 g·L⁻¹), metabolic yields (>78%) and overall ethanol productivities (0.57 g·L⁻¹.h⁻¹) in undetoxified StEx generated whole slurry hydrolysates derived from various lignocellulosic residues (Table 3). Despite its low specific xylose uptake rate and therefore low overall volumetric productivity, transformant TP1 achieved overall xylose consumption (88%) akin to RWB218, GS1.11-26, XH7, and LF1 with higher metabolic yields and ethanol concentrations. In essence, the transformant TP1 achieved ethanol yields similar to those achieved by S. cerevisiae 424A (LNH-ST) on highly fermentable ammonia fiber expansion (AFEX™) pretreated SCB hydrolysates and significantly higher than those achieved by the same strain on StEx-treated and undetoxified SCB whole slurry hydrolysates. CelluX™4 produced volumetric ethanol productivities that were 2-fold higher than TP1, while generating overall xylose consumption, metabolic yield, and ethanol concentrations that were higher than those demonstrated by S. cerevisiae strains RWB218, GS1.11-26, XH7, and LF1. Nonetheless, the high ethanol yield achieved by both TP1 and CelluX™4 in this work suggests that both strains are among the most promising xylose-fermenting yeast strains for the efficient conversion of both glucose and xylose in inhibitor-laden hydrolysates derived from autohydrolysis based pretreatment technologies, such as StEx.

2. Transformed CelluX™4 strains

Single Integrations

The TF and TA integrations did not have a pronounced effect on the CelluX™4 fermentation efficiency in 50% v/v SSL.HPLC samples of transformants with the highest weight loss were analyzed and the final ethanol titers and yields were similar to that of CelluX™4 (Table 4). Interestingly, the TA transformants showed a slightly higher xylose utilization than CelluX™4 control. The TF transformants xylose utilization seemed relatively unchanged (Table 4).

Overall, the first integration did not have such a pronounced effect and the data observed is similar to the CelluX™1 data. Also, the transformants lack the FPS1 gene knockout. The final pH of the TA and TF fermentations ranged between 4.4-4.5 and 4.35-4.45, respectively.

TABLE 4
The fermentation results of the TA and TF transformants
with the highest weight loss.

| | Used | Res. | Used | | Final | Final |
|---|---|---|---|---|---|---|
| | Sugar | Xyl. | Xyl | EtOH | Yield | Weight |
| | (g) | (g) | (g) | (g/L) | (g/g) | loss (g) |
| TF Control | 14.7 | 34.0 | 8.9 | 5.3 | 0.110 | 0.61 |
| CelluX ™4 | | | | | | |
| TF1 | 14.1 | 34.6 | 8.3 | 5.1 | 0.106 | 0.64 |
| TF3 | 14.4 | 34.4 | 8.5 | 5.4 | 0.112 | 0.62 |
| TF12 | 14.9 | 33.9 | 9.0 | 4.9 | 0.100 | 0.64 |
| TA Control | 13.9 | 34.8 | 8.1 | 4.2 | 0.086 | 0.52 |
| CelluX ™4 | | | | | | |
| TA5 | 15.4 | 33.4 | 9.5 | 4.7 | 0.097 | 0.55 |
| TA8 | 16.7 | 32.1 | 10.8 | 4.7 | 0.096 | 0.56 |
| TA12 | 14.9 | 33.8 | 9.1 | 3.2 | 0.066 | 0.55 |

Double Integrations

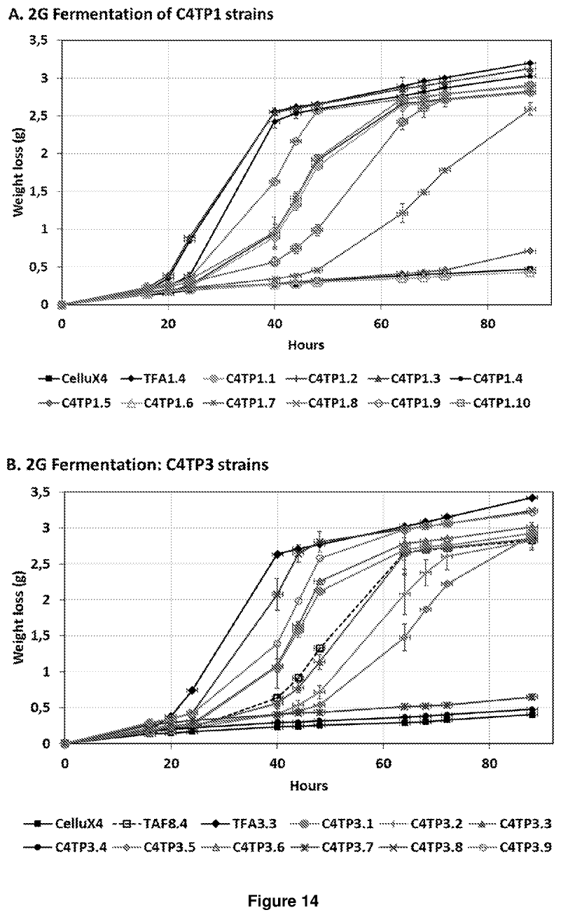

The CelluX™4-derived transformants with double integrations exhibited superior weight loss when compared to CelluX™4 reference strain (FIGS. 14A and 14B). This trend remained constant throughout the fermentation, indicating superior fermentation ability of transformants with double integrations. Interestingly, the TAF and TFA transformants performed relatively similar in terms of weight loss.

The HPLC data indicated that the TAF transformants had higher xylose utilization, with an increase of up to 68% when compared to the CelluX™4 control strain (Table 5), whereas the TFA transformants only displayed increment of up to 27% (Table 6). The sugar utilization is not reflected in the final ethanol titers, even though the ethanol titers for all transformants are slightly higher than that of CelluX™4. The fermentations were conducted with airlocks, which can be used to indicate the fermentation vigour. However, after 48-72 h the airlocks started developing negative space, beginning with the transformants. This could indicate increased 02 use, from either a possible diauxic shift, or the xylose being used during respiration.

TABLE 5
The fermentation results of the TAF transformants.

| | Used | Res. | Used | | | Final |
|---|---|---|---|---|---|---|
| | Sugar | Xyl. | Xyl. | EtOH | Yield | Weight |
| Strains | (g) | (g) | (g) | (g/L) | (g/g)_ | loss (g) |
| CelluX ™4 | 13.8 | 40.2 | 7.0 | 4.58 | 0.085 | 0.66 |
| TAF8.1 | 18.2 | 35.5 | 11.8 | 5.17 | 0.096 | 0.75 |
| TAF8.3 | 17.5 | 36.2 | 11.1 | 4.83 | 0.090 | 0.78 |
| TAF8.4 | 18.2 | 35.5 | 11.8 | 4.88 | 0.091 | 0.80 |

TABLE 6
The fermentation results of the TFA transformants.

| | | Res. | Used | | | Final |
|---|---|---|---|---|---|---|
| | Used | Xyl. | Xyl. | EtOH | Yield | Weight |
| Strains | Sugar (g) | (g) | (g) | (g/L) | (g/g) | loss (g) |
| CelluX ™4 | 16.4 | 39.8 | 8.2 | 4.5 | 0.081 | 0.68 |
| TFA1.2 | 16.8 | 39.3 | 8.57 | 4.87 | 0.087 | 0.79 |
| TFA1.4 | 17.8 | 38.3 | 9.60 | 5.07 | 0.090 | 0.75 |
| TFA1.5 | 18.4 | 37.7 | 10.2 | 5.05 | 0.090 | 0.76 |
| TFA3.3 | 18.6 | 37.5 | 10.4 | 4.96 | 0.088 | 0.75 |

Sugarcane Hydrolysate Fermentations

The 3^{rd} integration C4TP1 and C4TP3 transformants were screened in 50% w/w sugarcane hydrolysate fermentations under industrially relevant conditions. Fermentation commenced over a period of 88 h with three sample points in every 24 h period. Ten random C4TP1 transformants were selected as well as TFA1.4 (predecessor), and CelluX™4 was used as control strain. The TFA1.4 and C4TP1.3 transformants exhibited superior fermentation ability as both transformants commenced with fermentation within 24 h, and displayed higher weight loss after 88 h of fermentation when compared to all other transformants (FIG. 14A). The CelluX™4 control strain did not demonstrate any significant fermentation ability over the 88 h fermentation period, as did the C4TP1.6 transformant. Overall, nine out of the ten C4TP1 transformants outperformed the CelluX™4 control strain with weight loss ranging between 0.71-3.12 g versus 0.47 g loss of CelluX™4 (FIG. 14A).

Only nine C4TP3 transformants were randomly selected to include in fermentation screening, as well as TFA3.3 (predecessor), with CelluX™4 as a control strain. This was done to also include the TAF8.4 transformant in fermentation screening, as feedstock was limited. The TFA3.3 transformant exhibited the highest weight loss at 3.42 g, relative to the other transformants and control strain (FIG. 14B). The C4TP3.8 and C4TP3.9 transformants similarly exhibited high weight loss at 3.24 and 3.21 g weight loss, respectively, similarly to C4TP1.3 (3.12 g). The C4TP3 transformants performed in the same way as the C4TP1 transformants. However, in both fermentations, CelluX™4 demonstrated decreased fermentation ability, with less the 0.5 g weight loss over 88 h of fermentation (FIG. 14). Overall, the transformants exhibited a range of weight loss behavior but significantly outperformed the CelluX™4 parental strain in most instances, barring the C4TP1.6 and C4TP3.4 transformants (FIG. 14).

The medium composition and fermentation products were analyzed using HPLC analysis. Medium was composed of 59.0±1.85 g/L glucose, 30.4±0.93 g/L xylose, 1.34±0.12 g/L arabinose, 6.29±0.19 g/L acetic acid, 1.06±0.11 g/L formic acid, 0.12±0.01 g/L HMF and 1.23±0.11 g/L furfural, with pH of 5.2. Given the higher concentrations of microbial inhibitors, the xylose phenotype was of specific interest, as this heterologous pathway is more susceptible to inhibitor inhibition. Interestingly, all transformants barring C4TP3.4 demonstrated substantially higher xylose consumption than the CelluX™4 control strain that consumed <6% of the available xylose (Table 7, Table 8). In contrast, the TFA3.3 transformant consumed 75% of the available xylose, whereas the TFA1.4 transformant consumed 72.1%, a more than 10 fold increase in both instances. The TAF8.4 transformant, however, exhibited a lower xylose consumption at 30% (Table 8), a more than 2 fold decrease relative to the other 2^{nd} integration transformants. Also, the 2^{nd} integration transformants display higher xylose consumption when compared to the 3^{rd} integration transformants, as was observed previously during CelluX™1 strain development. The C4TP1 transformants are derived from TFA1.4 and exhibited xylose consumption ranging from 9.57-61.7%, whereas the C4TP3 transformants (derived from TFA3.3) exhibited xylose consumption ranging from 2.08-44.7% (Table 8).

TABLE 7
Fermentation kinetics of C4TP1 transformants in 50% w/w sugarcane
hydrolysate after 88 h.

| | | Xylose | | | | | | CO₂ |
|---|---|---|---|---|---|---|---|---|
| | Glucose | cons. | EtOH | EtOH yield | Productivity | Metabolic | | loss |
| Strains | cons. %. | % | (g/L) | (g/g) | g/L/h | yield % | pH | (g) |
| CelluX ™4 | 16.8 | 5.79 | 3.22 ± | 0.035 ± | 0.037 ± 0.01 | 52.5 | 4.90 | 0.47 |
| | | | 0.94 | 0.01 | | | | |
| TFA1.4 | 100 | 72.1 | 39.4 ± | 0.430 ± | 0.448 ± 0.01 | 93.1 | 5.73 | 3.20 |
| | | | 0.79 | 0.01 | | | | |
| C4TP1.1 | 100 | 40.4 | 33.2 ± | 0.362 ± | 0.377 ± 0.01 | 88.9 | 5.26 | 2.80 |
| | | | 0.85 | 0.01 | | | | |
| C4TP1.2 | 100 | 42.4 | 33.9 ± | 0.369 ± | 0.385 ± 0.01 | 90.0 | 5.41 | 2.82 |
| | | | 0.75 | 0.01 | | | | |
| C4TP1.3 | 100 | 61.7 | 35.2 ± | 0.383 ± | 0.400 ± 0.02 | 86.3 | 5.77 | 3.12 |
| | | | 1.70 | 0.02 | | | | |
| C4TP1.4 | 100 | 55.9 | 34.4 ± | 0.375 ± | 0.391 ± 0.02 | 86.4 | 5.53 | 3.03 |
| | | | 2.20 | 0.02 | | | | |
| C4TP1.5 | 32.8 | 14.0 | 6.51 ± | 0.071 ± | 0.074 ± 0.01 | 52.4 | 4.91 | 0.71 |
| | | | 1.06 | 0.01 | | | | |
| C4TP1.6 | 16.0 | 9.57 | 1.95 ± | 0.021 ± | 0.022 ± 0.00 | 30.6 | 4.95 | 0.43 |
| | | | 0.10 | 0.00 | | | | |
| C4TP1.7 | 98.4 | 31.3 | 30.2 ± | 0.329 ± | 0.344 ± 0.00 | 85.4 | 5.16 | 2.59 |
| | | | 0.23 | 0.00 | | | | |
| C4TP1.8 | 100 | 37.8 | 31.8 ± | 0.346 ± | 0.361 ± 0.01 | 86.1 | 5.17 | 2.89 |
| | | | 0.93 | 0.01 | | | | |
| C4TP1.9 | 100 | 45.7 | 32.2 ± | 0.351 ± | 0.366 ± 0.01 | 84.4 | 5.31 | 2.91 |
| | | | 0.52 | 0.01 | | | | |
| C4TP1.10 | 100 | 35.1 | 31.2 ± | 0.340 ± | 0.355 ± 0.01 | 85.5 | 5.18 | 2.82 |
| | | | 0.71 | 0.01 | | | | |

* Yield as total ethanol titre/total sugar
± standard deviation of triplicate values

The TFA3.3 and TFA1.4 transformants demonstrated high final ethanol titers at 40.1 g/L and 39.4 g/L ethanol, after 88 h of fermentation. The C4TP1 transformants ethanol titers ranged between 1.95 g/L-35.2 g/L ethanol (Table 7), whereas the C4TP3 transformants ranged between 1.82 g/L-34.7 g/L ethanol after 88 h of fermentation (Table 8). The CelluX™4 control strain produced <5.0 g/L ethanol. Overall, apart from the C4TP1.6 and C4TP3.7 transformants, the CelluX™4 control strain was outperformed by all other transformants in terms of ethanol titer, ethanol yield, volumetric productivity, xylose consumption and metabolic yield (Table 7, Table 8).

TABLE 8
Fermentation kinetics of C4TP3 transformants in 50% w/w sugarcane
hydrolysate after 88 h.

| | Glucose | Xylose | | | | | | CO₂ |
|---|---|---|---|---|---|---|---|---|
| | cons. | cons. | EtOH | EtOH | Productivity | Metabolic | | loss |
| Strains | %. | % | (g/L) | yield* (g/g) | g/L/h | yield % | pH | (g) |
| CelluX ™4 | 9.06 | 2.11 | 2.09 ± | 0.02 ± 0.00 | 0.024 ± 0.00 | 69.5 | 4.99 | 0.40 |
| | | | 0.24 | | | | | |
| TAF8.4 | 100 | 30.0 | 32.6 ± | 0.37 ± 0.00 | 0.371 ± 0.00 | 96.5 | 5.19 | 2.83 |
| | | | 0.39 | | | | | |
| TFA3.3 | 100 | 75.2 | 40.1 ± | 0.46 ± 0.01 | 0.456 ± 0.01 | 98.8 | 6.07 | 3.42 |
| | | | 0.71 | | | | | |
| C4TP3.1 | 100 | 38.2 | 33.1 ± | 0.38 ± 0.01 | 0.376 ± 0.01 | 94.5 | 5.28 | 2.93 |
| | | | 0.46 | | | | | |
| C4TP3.2 | 100 | 30.6 | 31.7 ± | 0.36 ± 0.00 | 0.361 ± 0.00 | 93.7 | 5.22 | 2.82 |
| | | | 0.31 | | | | | |
| C4TP3.3 | 100 | 30.5 | 31.1 ± | 0.36 ± 0.01 | 0.353 ± 0.01 | 91.7 | 5.20 | 2.86 |
| | | | 0.55 | | | | | |
| C4TP3.4 | 7.16 | 3.06 | 1.82 ± | 0.02 ± 0.00 | 0.021 ± 0.00 | 73.0 | 4.99 | 0.51 |
| | | | 0.01 | | | | | |
| C4TP3.5 | 100 | 29.7 | 31.6 ± | 0.36 ± 0.01 | 0.359 ± 0.01 | 93.6 | 5.30 | 2.90 |
| | | | 0.62 | | | | | |
| C4TP3.6 | 100 | 40.9 | 32.9 ± | 0.38 ± 0.00 | 0.374 ± 0.00 | 92.9 | 5.32 | 3.01 |
| | | | 0.43 | | | | | |
| C4TP3.7 | 10.8 | 7.27 | 2.76 ± | 0.03 ± 0.01 | 0.031 ± 0.01 | 66.9 | 4.99 | 0.65 |
| | | | 0.53 | | | | | |
| C4TP3.8 | 100 | 44.7 | 34.7 ± | 0.40 ± 0.02 | 0.394 ± 0.02 | 96.3 | 5.41 | 3.24 |
| | | | 1.81 | | | | | |
| C4TP3.9 | 100 | 40.3 | 33.6 ± | 0.39 ± 0.01 | 0.381 ± 0.01 | 94.9 | 5.32 | 3.21 |
| | | | 0.55 | | | | | |

* Yield as total ethanol titre/total sugar
± standard deviation of triplicate values

Xylose Phenotype Characterization

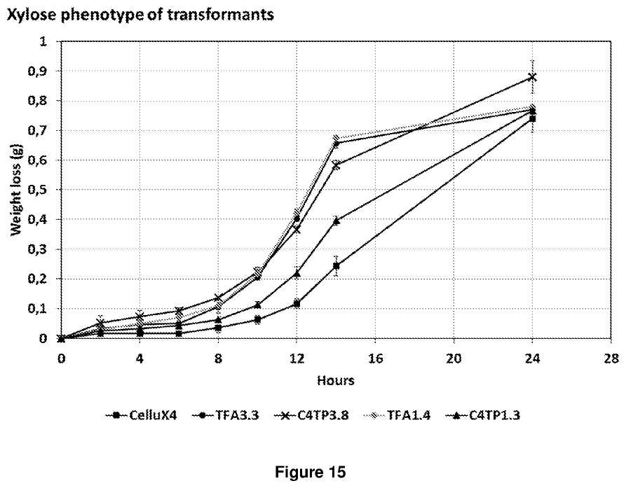

Maintaining the xylose phenotype is of key importance as this phenotype was previously shown to be negatively impacted by rational engineering for inhibitor resistance. The transformants were transferred from a glucose only medium into a xylose only medium to determine, (i) how fast transformants switched their metabolism from glucose to xylose, and (ii) how well strains utilize xylose. The transformants exhibited a faster switch between glucose to xylose metabolism when transferred from a glucose only-, to a xylose only medium in the first 6-14 h period of fermentation (p<0.05)(FIG. 15). The CelluX™4 control strain initially lagged behind, but after 24 h, there was no significant difference in weight loss between CelluX™4 and the transformants (p>0.05). Similarly, there was no difference in final biomass (OD₆₀₀) values (data not shown). Overall, when compared to the parental CelluX™4 strain, the transformants did not show substantial loss of xylose ability. This indicates that the transformants did not undergo significant loss of xylose ability during rational engineering of inhibitor resistance phenotype.

In the fermentation of StEx-treated and undetoxified SCB whole slurry hydrolysates, the transformant TP1 strain demonstrated high acetic acid resistance and significant furan aldehyde and phenolic aromatic aldehyde detoxification phenotypes, with furfural and 5-HMF assimilated to near completion within 24 h. Consequently, near-complete xylose consumption (88%), high ethanol concentrations (50 g L⁻¹), and high ethanol yields (0.436 g g⁻¹) were achieved after 144 h of fermenting the hydrolysates (Table 9), even in the presence of high acetic acid concentrations (˜8 g L⁻¹). Benchmarking the performance of TP1 in undetoxified whole slurry hydrolysates against literature reporting xylose-fermenting yeast strains, TP1 achieved similar overall xylose consumption but higher metabolic yields relative to some of the most promising recombinant yeast strains reported in the literature (Table 9). Although the metabolic yields, ethanol yields and volumetric productivities achieved by TP1 were bettered by the industrial recombinant strain CelluX™4, it is envisaged that if this strain were to be modified as described herein, even better yields and productivities would be achieved.

TABLE 9
Comparing literature reported yeast fermentation of undetoxified whole
slurry hydrolysates to S. cerevisiae TP-1, and S. cerevisiae CelluX ™4.

| | | Initial sugar | Glucose | Xylose | EtOH | | EtOH | | | | | |
|---|---|---|---|---|---|---|---|---|---|---|---|---|
| Yeast | | conc. | Cons | cons | conc. | Y_{P/S} | prod. | Metabolic | q [symbol] | q_{Glucose} | Q_{Xylene} | |
| Strain | Hydrolysate | (g · L⁻¹)^{ψ} | (%) | (%) | (g · L⁻¹) | (g · g⁻¹) * | (g · L⁻¹ · h⁻¹) [symbol] | Yield (%) ** | (g · L⁻¹ · h⁻¹) | (g · L⁻¹ · h⁻¹) | (g · L⁻¹ · h⁻¹) | Reference |
| S. cerevisiae | StEx-SCB whole | G: 80 | 100% | 88% | 50.0 | 0.44 | 0.347 | 89% | 0.063 | 0.406 | 0.034 | This study |
| TP-1 | slurry | X: 34 | | | | | | | | | | |
| | hydrolysate | | | | | | | | | | | |
| S. cerevisiae | StEx-SCB whole | G: 80 | 100% | 98% | 53.8 | 0.47 | 0.747 | 92% | 0.096 | 1.105 | 0.154 | This study |
| CelluX™4 | slurry | X: 34 | | | | | | | | | | |
| | hydrolysate | | | | | | | | | | | |
| S. cerevisiae | AFEX ™-SCB | G: 59 | 100% | 96% | 44.2 | 0.46 | 0.368 | 92% | 0.080 | 1.067 | 0.067 | Jansen et al. FEMS Yeast Res. |
| 424A (LNH-ST) | whole slurry | X: 37 | | | | | | | | | | 2017; 17. |
| | hydrolysate | | | | | | | | | | | |
| S. cerevisiae | AFEX ™ sugarcane | G: 5 [symbol] | 100% | 95% | 41.7 | 0.44 | 0.350 | 89% | 0.07 [symbol] | 1.033 | 0.061 | Jansen et al. FEMS Yeast Res. |
| 424A (LNH-ST) | CLM whole slurry | X: 35 | | | | | | | | | | 2017; 17. |
| | hydrolysate | | | | | | | | | | | |
| S. cerevisiae | StEx-SCB whole | G: 70 | 98% | 37% | 34.6 | 0. [symbol] 6 | 0.288 | 87% | 0.056 | 0.752 | 0.015 | Jansen et al. FEMS Yeast Res. |
| 424A (LNH-ST) | slurry hydrolysate | X: 26 | | | | | | | | | | 2017; 17. |
| S. cerevisiae | StEx sugarcane | G: 68 | 99% | 41% | 35.1 | 0. [symbol] 6 | 0.293 | 87% | 0.059 | 0.762 | 0.019 | Jansen et al. FEMS Yeast Res. |
| 424A (LNH-ST) | CLM whole slurry | X: 29 | | | | | | | | | | 2017; 17. |
| | hydrolysate | | | | | | | | | | | |
| S. cerevisiae | AFEX ™ corn | G: 60 | 100% | 58% | 39.0 | 0.43 | 0.325 | 9 [symbol] % | 0.083 | 0. [symbol] 54 | 0.037 | Jeffries T W. Curr. Opin. |
| GLBRC [symbol] | stover | X: 30 | | | | | | | | | | Biotechnol. 2006. p. 320-326. |
| | hydrolysate | | | | | | | | | | | |
| S. cerevisiae | LHW corn cobs | G: 2 | 100% | 99% | 7.7 | 0.28 | 0.107 | 5 [symbol] % | N/R | N/R | N/R | Vilela [symbol] de F. et al. AMB Express. |
| MEC1122 | pretreatment | X: 26 | | | | | | | | | | 2015; 5 |
| | liquor | | | | | | | | | | | |
| S. cerevisiae | StEx wheat straw | G: 50 | 99% | 88% | 38.1 | 0.47 | 0.693 | 99% | N/R | N/R | N/R | Demeke M M et al. Biotechnol |
| RWB 218 | hydrolysate | X: 20 | | | | | | | | | | Biofuels, 2013; 6 [symbol] 9. |
| S. cerevisiae | Dilute Acid spruce | G: 16 | 100% | 100% | 16 | 0.43 | 0.16 | 80% | 0.005 | 0.021 | 0.005 | EP 3 108 016 |
| TMB 3400 | hydrolysate [symbol] | X: 7 | | | | | | | | | | |
| S. cerevisiae | SO [symbol] —StEx spruce | G: 62 | 100% | 86% | 39.0 | 0.41 | 0.361 | 84% | N/R | N/R | N/R | Cunha et al. Appl Microbiol |
| GS1.11-26 | pretreatment | X: 18 | | | | | | | | | | Biotechnol. Applied Microbiology |
| | liquor [symbol] | | | | | | | | | | | and Biotechnology; 2019; 103: |
| | | | | | | | | | | | | 159-175. |
| S. cerevisiae | StEx corn stover | G: 78 | 99% | 80% | 41.5 | 0. [symbol] 9 | 0.569 | 78% | 0.146 | 1.67 | 0.124 | Feng Q. et al. Yang S, editor. PLoS |
| XH7 | hydrolysate | X: 39 | | | | | | | | | | One. Public Library of Science; |
| | | | | | | | | | | | | 2018; 13: e0195653. |
| S. cerevisiae | StEx corn stover | G: 80 | 99% | 89% | 49.0 | 0.41 | 1.021 | 84% | 0.254 | 1.62 | 0.181 | Feng et al. |
| LF1 | hydrolysate | X: 40 | | | | | | | | | | |

^{ψ}G: glucose,
X: Xylose;
^{ϕ}volumetric ethanol productivity;
^{†} Hydrolysate also composed of fermentable 4 g · L⁻¹ galactose and 10 g · L⁻¹ mannose;
^{‡} Hydrolysate also composed of 15 g · L⁻¹ fermentable mannose and supplemented with 50 g · L⁻¹ synthetic glucose.
* Ethanol Yield: ethanol produced relative to the available sugars in the hydrolysate;
** Metabolic yield: ethanol produced relative to the theoretical maximum ethanol based on consumed sugars in the hydrolysate.
CLM—Cane leaf matter:
LHW—Liquid hot water:
AFEX ™—ammonia fiber expansion:
StEx—steam explosion:
N/R—not reported [symbol]
indicates data missing or illegible when filed

1. A method of producing a modified Saccharomyces cerevisiae yeast strain with enhanced resistance to furans, phenolics and weak acids, the method comprising the step of integrating at least one copy of a TAL1 gene and at least one copy of two or more of a FDH1 gene, ADH6 gene and ARI1 gene into the S. cerevisiae genome, so that the modified yeast strain overexpresses these genes relative to a S. cerevisiae yeast strain that has not been modified in the same manner.

2. The method according to claim 1, which further comprises integrating at least one copy of either or both of a PAD1 gene and ICT1 gene into the S. cerevisiae genome.

3. The method according to claim 1, which further comprises at least partially or completely deleting the FPS1 gene in the S. cerevisiae genome.

4. The method according to claim 1, wherein each of the integrated genes is under the control of a constitutive promoter.

5. The method according to claim 1, wherein the strain is a S. cerevisiae strain with xylose utilizing capacity.

6. The method according to claim 1, wherein:
at least one of each of the TAL1, FDH1 and ARI1 genes are integrated into the S. cerevisiae genome
at least one of each of the TAL1, FDH1 and ADH6 genes are integrated into the S. cerevisiae genome; or
at least one of each of the TAL1, ADH6 and ARI1 genes are integrated into the S. cerevisiae genome.

7-8. (canceled)

9. The method according claim 1, wherein at least one of each of the TAL1, FDH1, ARI1 and ADH6 genes are integrated into the S. cerevisiae genome.

10. The method according to claim 1, wherein:
at least one of each of the TAL1, FDH1, ARI1 genes and either or both of PAD1 and ICT1 genes are integrated into the S. cerevisiae genome;
at least one of each of the TAL1, FDH1, ADH6 genes and either or both of PAD1 and ICT1 genes; or
at least one of each of the TAL1, FDH1, ARI1, ADH6 genes and either or both of PAD1 and ICT1 genes are integrated into the S. cerevisiae genome.

11-13. (canceled)

14. The method according to claim 6, wherein the genes are integrated into the S. cerevisiae genome in any order.

15. The method according to claim 6, wherein the genes are integrated into the S. cerevisiae genome in the order as listed.

16. A double gene expression cassette comprising:
(a) TAL1 and FDH1 genes;
(b) ARI1 and ADH6 genes;
(c) PAD1 and ICT1 genes;
(d) TAL1 and ADH6 genes; or
(e) AR/1 and FDH1 genes.

17-20. (canceled)

21. The double gene expression cassette according to claim 16, wherein the TAL1, ARI1 and PAD1 genes are under the control of a PGK1 gene promoter.

22. The double gene expression cassette according to claim 16, wherein the FDH1, ADH6 and ICT1 genes are under the control of an ENO1 gene promoter.

23. A Saccharomyces cerevisiae yeast strain which has been modified to overexpress at least a TAL1 gene and two or more of FDH1, ADH6 and ARI1 genes relative to an unmodified strain, wherein the modified strain has increased resistance to furans, phenolics and weak acids compared to an unmodified strain.

24. The modified yeast strain according to claim 23, which further overexpress one or both of PAD1 and ICT1 genes relative to an unmodified strain.

25. The modified yeast strain according to claim 23, wherein the FPS1 gene is partially or completely deleted in the modified strain.

26. The modified yeast strain according to claim 23, which further comprises multiple integrated copies of the genes which are overexpressed.

27. The modified yeast strain according to claim 23, wherein each of the integrated genes is under the control of a constitutive promoter.

28. The modified yeast strain according to claim 23, which has xylose utilizing capacity.

29. The modified yeast strain according to claim 23, which comprises integrated copies of at least the following genes:
a) TAL1, FDH1 and ARI1;
b) TAL1, FDH1 and ADH6;
c) TAL1, FDH1, ARI1 and ADH6;
d) TAL1, FDH1, ARI1 and either or both of PAD1 and ICT1;
e) TAL1 FDH1, ADH6 and either or both of PAD1 and ICT1;
f) TAL1, FDH1, ARI1, ADH6 and either or both of PAD1 and ICT1;
g) TAL1, ADH6 and ARI1;
h) TAL1, ADH6 and FDH1;
i) TAL1, ADH6, ARI1 and FDH1; or
j) TAL1, ADH6, ARI1, FDH1 and either or both of PAD1 and ICT1.

30-32. (canceled)
